(19) United States
(12) Patent Application Publication
(10) Pub. No.: US 2023/0089559 A1
(43) Pub. Date: Mar. 23, 2023

(54) COMPOSITIONS AND METHODS RELATING TO AGING SKIN

(71) Applicant: Alastin Skincare, Inc., Carlsbad, CA (US)

(72) Inventors: Alan David WIDGEROW, Carlsbad, CA (US); John A. GARRUTO, Carlsbad, CA (US)

(73) Assignee: Alastin Skincare, Inc., Carlsbad, CA (US)

(21) Appl. No.: 17/991,532

(22) Filed: Nov. 21, 2022

Related U.S. Application Data
(63) Continuation of application No. PCT/US2021/033422, filed on May 20, 2021.
(60) Provisional application No. 63/028,342, filed on May 21, 2020.

(51) Int. Cl. A61K 8/64; A61K 8/55; A61K 8/9706; A61K 8/9783; A61K 8/60; A61Q 19/08

(52) U.S. Cl. CPC A61K 8/64; A61K 8/553; A61K 8/9706; A61K 8/9783; A61K 8/60; A61Q 19/08

(57) ABSTRACT

Compositions and methods for promoting skin restoration of aging skin are provided herein. Compositions and methods described herein may result in elastin and/or collagen stimulation, extracellular matrix (ECM) recycling, anti-inflammatory effects, or combinations thereof.

### CROSS-REFERENCE

This application is a continuation of International Patent Application No. PCT/US2021/033422 filed May 20, 2021, which application claims the benefit of U.S. Provisional Patent Application No. 63/028,342 filed on May 21, 2020, both of which are incorporated herein by reference in their entireties.

### BACKGROUND

Skin changes with age becoming thinner, less elastic, and more prone to bruising. This is due in part a result of atrophy and alteration in collagen, elastin, and components of the extracellular matrix. Compositions are needed to counter these effects associated with aging skin.

### BRIEF SUMMARY

An aspect described herein are methods for promoting restoration of aging skin comprising administering to a skin region of an individual with dermatoporosis a composition comprising a tripeptide-1, a hexapeptide-12, a hexapeptide-11, and a tetrapeptide-2. In one feature, the tripeptide-1 is present at 1-10 ppm. In one feature, the tripeptide-1 is present at about 0.0001 wt. % to about 1 wt. %. In one feature, the tripeptide-1 is present at about 1 wt. % to about 6 wt. %. In one feature, the tripeptide-1 comprises palmitoyl tripeptide-1, myristoyl tripeptide-1, or a combination thereof. In one feature, the hexapeptide-12 comprises palmitoyl hexapeptide-12, myristoyl hexapeptide-12, or a combination thereof. In one feature, the hexapeptide-12 is present at 1-10 ppm. In one feature, the hexapeptide-12 is present at about 0.0001 wt. % to about 1 wt. %. In one feature, the hexapeptide-12 is present at about 0.25 wt. % to about 4 wt. %. In one feature, the hexapeptide-11 is present at 50-150 ppm. In one feature, the hexapeptide-11 is present at about 0.005 wt. % to about 0.02 wt. %. In one feature, the composition further comprises hexapeptide-38. In one feature, the hexapeptide-38 is acetyl hexapeptide-38. In one feature, the hexapeptide-38 is present at about 0.0001 wt. % to about 1 wt. %. In one feature, the method further comprises phosphatidylserine. In one feature, the phosphatidylserine is present in a range of about 0.005 wt. % to about 0.1 wt. %. In one feature, the phosphatidylserine is present at no more than 5.0 wt %. In one feature, the composition further comprises hydroxymethoxyphenyl decanone. In one feature, the composition further comprises dill extract. In one feature, the composition further comprises palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or combinations thereof. In one feature, the aging skin is more prone to bruising, is subject to photodamage, is exposed to solar radiation, is exposed to actinic damage, or combinations thereof. In one feature, the composition is administered prior to appearance of a bruise, an aging spot, or a wrinkle. In one feature, the composition is administered after appearance of a bruise, an aging spot, or a wrinkle. In one feature, the composition is administered 1, 2, 3, 4, 5, 6, 7, or 8 times a day. In one feature, the individual is a human.

An aspect described herein are methods for promoting restoration of aging skin comprising administering to a skin region of an individual with dermatoporosis a first composition comprising a first tripeptide-1, a first hexapeptide-12, a first hexapeptide-11, and a first tetrapeptide-2, and a second composition comprising a second tripeptide-1, a second hexapeptide-12, a second hexapeptide-11, a second tetrapeptide-2, and a hexapeptide-38. In one feature, the first tripeptide-1, the second tripeptide-1, or both is present at 1-10 ppm. In one feature, the first tripeptide-1, the second tripeptide-1, or both is present at about 0.0001 wt. % to about 1 wt. %. In one feature, the first tripeptide-1, the second tripeptide-1, or both is present at about 1 wt. % to about 6 wt. %. In one feature, the first tripeptide-1, the second tripeptide-1, or both comprises palmitoyl tripeptide-1, myristoyl tripeptide-1, or a combination thereof In one feature, the first hexapeptide-12, the second hexapeptide-12, or both comprises palmitoyl hexapeptide-12, myristoyl hexapeptide-12, or a combination thereof. In one feature, the first hexapeptide-12, the second hexapeptide-12, or both is present at 1-10 ppm. In one feature, the first hexapeptide-12, the second hexapeptide-12, or both is present at about 0.0001 wt. % to about 1 wt. %. In one feature, the first hexapeptide-12, the second hexapeptide-12, or both is present at about 0.25 wt. % to about 4 wt. %. In one feature, the first hexapeptide-11, the second hexapeptide-11, or both is present at 50-150 ppm. In one feature, the first hexapeptide-11, the second hexapeptide-11, or both is present at about 0.005 wt. % to about 0.02 wt. %. In one feature, the hexapeptide-38 is present at about 0.0001 wt. % to about 1 wt. %. In one feature, the first composition, the second composition, or both further comprises phosphatidylserine. In one feature, the phosphatidylserine is present in a range of about 0.005 wt. % to about 0.1 wt. %. In one feature, the phosphatidylserine is present at no more than 5.0 wt %. In one feature, the first composition, the second composition, or both composition further comprises hydroxymethoxyphenyl decanone. In one feature, the first composition further comprises dill extract. In one feature, the first composition, the second composition, or both further comprises palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or combinations thereof. In one feature, the aging skin is more prone to bruising, is subject to photodamage, is exposed to solar radiation, is exposed to actinic damage, or combinations thereof. In one feature, the first composition, the second composition, or both is administered prior to appearance of a bruise, an aging spot, or a wrinkle. In one feature, the first composition, the second composition, or both is administered after appearance of a bruise, an aging spot, or a wrinkle. In one feature, the first composition is administered prior to appearance of a bruise, an aging spot, or a wrinkle and the second composition is administered after appearance of the bruise, the aging spot, or the wrinkle. In one feature, the first composition, the second composition, or both is administered 1, 2, 3, 4, 5, 6, 7, or 8 times a day. In one feature, the individual is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows images of bruising resolution following administration of the topical products described herein.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Compositions

Aging skin can be subject to atrophy and alterations in collagen and elastin. The extracellular matrix (ECM) can also be subject to thinning and atrophy, resulting in the skin being more vulnerable and sensitive to trauma. With this trauma, extravasation of red blood cells into the dermis results in the hemosiderin deposits in the interstitial space manifesting as bruising and on occasion permanent pigmentary changes to the skin. This occurs in a background of minimal inflammatory reaction within the dermal tissue with resulting deficiencies in the macrophage response delaying absorption of the blood products. Accordingly, compositions are needed for reversing the effects of aging skin.

Described herein are compositions and methods for restoring dermal integrity and vessel support, recycle the ECM, increasing collagen, elastin and glycosaminoglycans (GAGS) such as hyaluronic acid (HA), replacing waste products with new matrix components, and combinations thereof. Compositions and methods as described herein can improve macrophage function. Compositions and methods as described herein may further stimulate elastin and/or collagen production, intrinsic hyaluronic acid production, or reduce inflammation.

Peptides

Peptides as described herein, in some embodiments, restore dermal integrity and vessel support, recycle the ECM, increase collagen, elastin and glycosaminoglycans (GAGS) such as hyaluronic acid (HA), and replace waste products with new matrix components. In some embodiments, peptides as described herein improve macrophage function. In some embodiments, tripeptide-1 results in elastin and/or collagen stimulation, extracellular matrix (ECM) recycling, anti-inflammatory effects, or combinations thereof. In some embodiments, hexapeptide-12 draws in newly produced elastin and stimulates angiogenesis and neovascularization. In some embodiments, acetyl tetrapeptide-2 stimulates fibroblasts to produce elastin.

The peptide combinations of the embodiments can be employed in various types of compositions. Topical compositions including a dipeptide, tripeptide, or tetrapeptide, and a pentapeptide, hexapeptide, or heptapeptide in combination with at least one excipient, are provided. In some embodiments, topical compositions comprise one or more tripeptides, one or more tetrapeptides, and one or more hexapeptides. In some embodiments, a tripeptide of the one or more tripeptides is tripeptide-1. In some embodiments, a tetrapeptide of the one or more tetrapeptides is tetrapeptide-2. In some embodiments, a hexapeptide of the one or more hexapeptides is hexapeptide-12. In some embodiments, a hexapeptide of the one or more hexapeptides is hexapeptide-11. In some embodiments, a hexapeptide of the one or more hexapeptides is hexapeptide-38. In some embodiments, the topical composition comprises tripeptide-1, tetrapeptide-2, hexapeptide-12, and hexapeptide-11. In some embodiments, the topical composition comprises tripeptide-1, tetrapeptide-2, hexapeptide-12, hexapeptide-11, and hexapeptide-38. In some embodiments, the composition comprises a tripeptide-1, a hexapeptide-12 and a hexapeptide-11. In some embodiments, the topical composition further comprises a tetrapeptide. In some embodiments, the tetrapeptide is tetrapeptide-2. In some embodiments, the topical composition comprises tripeptide-1, tetrapeptide-2, and hexapeptide-12.

Compositions as described herein comprise a varying concentration of peptide. In some instances, a peptide is present at about 50 ppm or less to 1000, 5000, 10000, 50000, 100000, 500000 ppm or more, e.g., 100 ppm of the peptide. In some instances, a peptide is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (ug/mL). In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide is present from about 0.01% to about 10%, about 0.01% to about 0.02%, about 0.01% to about 0.03%, about 0.01% to about 0.04%, about 0.01% to about 0.05%, about 0.01% to about 0.1%, about 1% to about 5%, or about 1% to about 10% by weight (wt. %).

Compositions as described herein, in some embodiments, comprise a plurality of peptides. In some instances, a peptide of the plurality of peptides is present at about 50 ppm or less to 1000, 5000, 10000, 50000, 100000, 500000 ppm or more, e.g., 100 ppm of the peptide, or any other suitable amount. In some instances, a peptide of the plurality of peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, a peptide of the plurality of peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide of the plurality of peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (ug/mL). In some instances, a peptide of the plurality of peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide of the plurality of peptides is present from about 0.01% to about 10%, about 0.01% to about 0.02%, about 0.01% to about 0.03%, about 0.01% to about 0.04%, about 0.01% to about 0.05%, about 0.01% to about 0.1%, about 1% to about 5%, or about 1% to about 10% by weight (wt. %). In some embodiments, a peptide of the plurality of peptides is provided at least or about 0.00001%, 0.0003%, 0.0005%, 0.001%, 0.001%, 0.005%, 0.0055%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, a peptide of the plurality of peptides is provided in a range of about 0.25% to about 10%, about 0.5% to about 8%, about 0.75% to about 6%, or about 1% to about 4% by weight. In some embodiments, each peptide of the plurality of peptides is provided in a range of about 0.001% to about 6%, about 0.002% to about 4%, about 0.01% to about 3%, or about 0.02% to about 2% by weight. In some embodiments, the peptide is tripeptide-1, hexapeptide-12, hexapeptide-11, hexapeptide-38, tetrapeptide-2, or combinations thereof.

In some embodiments, the tripeptide-1 is provided at least or about 0.00001%, 0.0003%, 0.0005%, 0.001%, 0.001%, 0.005%, 0.0055%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the tripeptide-1 is provided in a range of about 0.25% to about 10%, about 0.5% to about 8%, about 0.75% to about 6%, or about 1% to about 4% by weight. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tripeptide-1 is provided in a range of about 0.25 to about 10, about 0.5 to about 8, about 1 to about 6, or about 2 to about 4 ppm. In some embodiments, the tripeptide-1 is provided in a range of about 1 to about 10 ppm. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (ug/mL). In some embodiments, the tripeptide-1 is provided in a range of about 0.25 to about 10, about 0.5 to about 8, about 1 to about 6, or about 2 to about 4 microgram per milliliter.

In some embodiments, the tripeptide-1 is provided at least or about 30, 40, 50, 60, 70, 80, 90, 100, 200, 300, 400, 500, 600, 700, 800, 900, 1000, 2000, or more than 2000 microgram (ug). In some embodiments, the tripeptide-1 is provided at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, or more than 2000 milligrams (mg). In some embodiments, the tripeptide-1 is provided in a range of about 30 to about 2000 ug. In some embodiments, the tripeptide-1 is provided in a range of about 40 to about 1000, about 50 to about 900, about 60 to about 800, about 70 to about 700, about 80 to about 600, or about 90 to about 500 ug. In some embodiments, the tripeptide-1 is provided at least or about 150 ug. In some embodiments, the tripeptide-1 is provided at least or about 450 ug. In some embodiments, the tripeptide-1 is provided in a range of about 30 to about 2000 mg. In some embodiments, the tripeptide-1 is provided in a range of about 40 to about 1000, about 50 to about 900, about 60 to about 800, about 70 to about 700, about 80 to about 600, or about 90 to about 500 mg. In some embodiments, the tripeptide-1 is provided at least or about 150 mg. In some embodiments, the tripeptide-1 is provided at least or about 450 mg.

In some embodiments, the hexapeptide-12 is provided at least or about 0.00001%, 0.0003%, 0.0005%, 0.001%, 0.001%, 0.005%, 0.0055%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the hexapeptide-12 is provided in a range of about 0.25% to about 10%, about 0.5% to about 8%, about 0.75% to about 6%, or about 1% to about 4% by weight. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the hexapeptide-12 is provided in a range of about 1 to about 10 ppm. In some embodiments, the hexapeptide-12 is provided in a range of about 0.25 to about 10, about 0.5 to about 8, about 1 to about 6, or about 2 to about 4 ppm. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (ug/mL). In some embodiments, the hexapeptide-12 is provided in a range of about 0.25 to about 10, about 0.5 to about 8, about 1 to about 6, or about 2 to about 4 microgram per milliliter.

In some embodiments, the hexapeptide-12 is provided at least or about 30, 40, 50, 60, 70, 80, 90, 100, 200, 300, 400, 500, 600, 700, 800, 900, 1000, 2000, or more than 2000 microgram (ug). In some embodiments, the hexapeptide-12 is provided at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, or more than 2000 milligrams (mg). In some embodiments, the hexapeptide-12 is provided in a range of about 30 to about 2000 ug. In some embodiments, the hexapeptide-12 is provided in a range of about 40 to about 1000, about 50 to about 900, about 60 to about 800, about 70 to about 700, about 80 to about 600, or about 90 to about 500 ug. In some embodiments, the hexapeptide-12 is provided at least or about 150 ug. In some embodiments, the hexapeptide-12 is provided at least or about 450 ug. In some embodiments, the hexapeptide-12 is provided in a range of about 30 to about 2000 mg. In some embodiments, the hexapeptide-12 is provided in a range of about 40 to about 1000, about 50 to about 900, about 60 to about 800, about 70 to about 700, about 80 to about 600, or about 90 to about 500 mg. In some embodiments, the hexapeptide-12 is provided at least or about 150 mg. In some embodiments, the hexapeptide-12 is provided at least or about 450 mg.

In some embodiments, the hexapeptide-11 is provided at least or about 0.00001%, 0.0003%, 0.0005%, 0.001%, 0.001%, 0.005%, 0.0055%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, 20%, 30%, 40%, 50%, 60%, 70%, 80%, 90% or more than 90% by weight (wt. %). In some embodiments, the hexapeptide-11 is provided in a range of about 0.25% to about 10%, about 0.5% to about 8%, about 0.75% to about 6%, or about 1% to about 4% by weight. In some embodiments, the hexapeptide-11 is provided in a range of about 0.001% to about 6%, about 0.002% to about 4%, about 0.01% to about 3%, or about 0.02% to about 2%. In some embodiments, the hexapeptide-11 is provided in a range of about 0.005% to about 0.02% by weight. In some embodiments, the hexapeptide-11 is provided at least or about 5, 10, 20, 25, 50, 75, 100, 150, 200, 250, or more than 250 ppm. In some embodiments, the hexapeptide-11 is provided in a range of about 25 to about 250, about 50 to about 200, or about 75 to about 150 ppm. In some embodiments, the hexapeptide-11 is provided in a range of about 10 to about 100 ppm. In some embodiments, the hexapeptide-11 is provided at least or about 5, 10, 20, 25, 50, 75, 100, 150, 200, 250, or more than 250 microgram per milliliter (ug/mL). In some embodiments, the hexapeptide-11 is provided in a range of about 25 to about 250, about 50 to about 200, or about 75 to about 150 microgram per milliliter.

In some embodiments, the hexapeptide-11 is provided at least or about 30, 40, 50, 60, 70, 80, 90, 100, 200, 300, 400, 500, 600, 700, 800, 900, 1000, 2000, or more than 2000 microgram (ug). In some embodiments, the hexapeptide-11 is provided at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, or more than 2000 milligrams (mg). In some embodiments, the hexapeptide-11 is provided in a range of about 30 to about 2000 ug. In some embodiments, the hexapeptide-11 is provided in a range of about 40 to about 1000, about 50 to about 900, about 60 to about 800, about 70 to about 700, about 80 to about 600, or about 90 to about 500 ug. In some embodiments, the hexapeptide-11 is provided at least or about 150 ug. In some embodiments, the hexapeptide-11 is provided at least or about 450 ug. In some embodiments, the hexapeptide-11 is provided in a range of about 30 to about 2000 mg. In some embodiments, the hexapeptide-11 is provided in a range of about 40 to about 1000, about 50 to about 900, about 60 to about 800, about 70 to about 700, about 80 to about 600, or about 90 to about 500 mg. In some embodiments, the hexapeptide-11 is provided at least or about 150 mg. In some embodiments, the hexapeptide-11 is provided at least or about 450 mg.

In some embodiments, the hexapeptide-38 is provided at least or about 0.00001%, 0.0003%, 0.0005%, 0.001%, 0.001%, 0.005%, 0.0055%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, 20%, 30%, 40%, 50%, 60%, 70%, 80%, 90% or more than 90% by weight (wt. %). In some embodiments, the hexapeptide-38 is provided in a range of about 0.25% to about 10%, about 0.5% to about 8%, about 0.75% to about 6%, or about 1% to about 4% by weight. In some embodiments, the hexapeptide-38 is provided in a range of about 0.001% to about 6%, about 0.002% to about 4%, about 0.01% to about 3%, or about 0.02% to about 2%. In some embodiments, the hexapeptide-38 is provided in a range of about 0.005% to about 0.02% by weight. In some embodiments, the hexapeptide-38 is provided at least or about 0.25, 0.5, 0.75, 1, 1.5, 2, 2.5, 3, 3.5, 4, 5, 10, 20, 25, 50, 75, 100, 150, 200, 250, or more than 250 ppm. In some embodiments, the hexapeptide-38 is provided in a range of about 25 to about 250, about 50 to about 200, or about 75 to about 150 ppm. In some embodiments, the hexapeptide-38 is provided at least or about 5, 10, 20, 25, 50, 75, 100, 150, 200, 250, or more than 250 microgram per milliliter (ug/mL). In some embodiments, the hexapeptide-38 is provided in a range of about 25 to about 250, about 50 to about 200, or about 75 to about 150 microgram per milliliter. In some embodiments, the hexapeptide-38 is acetyl hexapeptide-38.

In some embodiments, the hexapeptide-38 is provided at least or about 30, 40, 50, 60, 70, 80, 90, 100, 200, 300, 400, 500, 600, 700, 800, 900, 1000, 2000, or more than 2000 microgram (ug). In some embodiments, the hexapeptide-38 is provided at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, or more than 2000 milligrams (mg). In some embodiments, the hexapeptide-38 is provided in a range of about 30 to about 2000 ug. In some embodiments, the hexapeptide-38 is provided in a range of about 40 to about 1000, about 50 to about 900, about 60 to about 800, about 70 to about 700, about 80 to about 600, or about 90 to about 500 ug. In some embodiments, the hexapeptide-38 is provided at least or about 150 ug. In some embodiments, the hexapeptide-38 is provided at least or about 450 ug. In some embodiments, the hexapeptide-38 is provided in a range of about 30 to about 2000 mg. In some embodiments, the hexapeptide-38 is provided in a range of about 40 to about 1000, about 50 to about 900, about 60 to about 800, about 70 to about 700, about 80 to about 600, or about 90 to about 500 mg. In some embodiments, the hexapeptide-38 is provided at least or about 150 mg. In some embodiments, the hexapeptide-38 is provided at least or about 450 mg.

In some embodiments, the tetrapeptide-2 is provided at least or about 0.00001%, 0.0003%, 0.0005%, 0.001%, 0.001%, 0.005%, 0.0055%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the tetrapeptide-2 is provided in a range of about 0.25% to about 10%, about 0.5% to about 8%, about 0.75% to about 6%, or about 1% to about 4% by weight. In some embodiments, the tetrapeptide-2 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tetrapeptide-2 is provided in a range of about 0.25 to about 10, about 0.5 to about 8, about 1 to about 6, or about 2 to about 4 ppm. In some embodiments, the tetrapeptide-2 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (ug/mL). In some embodiments, the tetrapeptide-2 is provided in a range of about 0.25 to about 10, about 0.5 to about 8, about 1 to about 6, or about 2 to about 4 microgram per milliliter.

In some embodiments, the tetrapeptide-2 is provided at least or about 30, 40, 50, 60, 70, 80, 90, 100, 200, 300, 400, 500, 600, 700, 800, 900, 1000, 2000, or more than 2000 microgram (ug). In some embodiments, the tetrapeptide-2 is provided at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, or more than 2000 milligrams (mg). In some embodiments, the tetrapeptide-2 is provided in a range of about 30 to about 2000 ug. In some embodiments, the tetrapeptide-2 is provided in a range of about 40 to about 1000, about 50 to about 900, about 60 to about 800, about 70 to about 700, about 80 to about 600, or about 90 to about 500 ug. In some embodiments, the tetrapeptide-2 is provided at least or about 150 ug. In some embodiments, the tetrapeptide-2 is provided at least or about 450 ug. In some embodiments, the tetrapeptide-2 is provided in a range of about 30 to about 2000 mg. In some embodiments, the tetrapeptide-2 is provided in a range of about 40 to about 1000, about 50 to about 900, about 60 to about 800, about 70 to about 700, about 80 to about 600, or about 90 to about 500 mg. In some embodiments, the tetrapeptide-2 is provided at least or about 150 mg. In some embodiments, the tetrapeptide-2 is provided at least or about 450 mg.

In example embodiments, a weight ratio for the first peptide to the second peptide in a topical composition is 1 part first peptide to 0.2 to 10 parts second peptide, 1 to 10 parts second peptide, 1 to 8 parts second peptide, or 1 to 5.5 parts second peptide. The following nomenclature is employed herein to refer to various amino acids: Alanine (also referred to herein as “Ala” or “A”), Arginine (also referred to herein as “Arg” or “R”), Asparagine (also referred to herein as “Asn” or “N”), Aspartic acid (also referred to herein as “Asp” or “D”), Cysteine (also referred to herein as “Cys” or “C”), Glutamic acid (also referred to herein as “Glu” or “E”), Glutamine (also referred to herein as “Gln” or “Q”), Glycine (also referred to herein as “Gly” or “G”), Histidine (also referred to herein as “His” or “H”), Isoleucine (also referred to herein as “Ile” or “I”), Leucine (also referred to herein as “Leu” or “L”), Lysine (also referred to herein as “Lys” or “K”), Methionine (also referred to herein as “Met” or “M”), Phenylalanine (also referred to herein as “Phe” or “F”), Proline (also referred to herein as “Pro” or “P”), Serine (also referred to herein as “Ser” or “S”), Threonine (also referred to herein as “Thr” or “T”), Tryptophan (also referred to herein as “Trp” or “W”), Tyrosine (also referred to herein as “Tyr” or “Y”), Valine (also referred to herein as “Val” or “V”).

In some embodiments, the first peptide is a dipeptide. Suitable dipeptides include but are not limited to those having the following sequence of amino acids: KK, KP, CK, KC, KT, DF, NF, VW, YR, or TT. In some embodiments, the dipeptide has the following amino acid sequence: KV. In other embodiments, the first peptide is a tripeptide. Suitable tripeptides include but are not limited to those having the following sequence of amino acids: HGG, RKR, GHK, GKH, GGH, GHG, KFK, or KPK. In some embodiments, the tripeptide has the following amino acid sequence: KVK. In some embodiments, the first peptide is a tetrapeptide. Suitable tetrapeptides include but are not limited to those having the following sequence of amino acids: GQPR, KTFK, AQTR, or RSRK. In some embodiments, the tetrapeptide has the following sequence of amino acids: KDVY. In some embodiments, the second peptide is a pentapeptide. Suitable pentapeptides include but are not limited to those having the following sequence of amino acids: KTTKS, YGGFX, or KLAAK. In some embodiments, the second peptide is a hexapeptide. Suitable hexapeptides include but are not limited to those having the following sequence of amino acids: VGVAPG or GKTTKS. In some embodiments, the hexapeptide has the following sequence of amino acids: FVAPFP. In some embodiments, the second peptide is a heptapeptide. Suitable heptapeptides include but are not limited to one having an amino acid sequence RGYYLLE, or Heptapeptide-6 (a pro-sirtuin peptide). The compositions may include two or more peptides, e.g., two dipeptides and one pentapeptide; one tripeptide and one hexapeptide; one dipeptide, one tripeptide, and one heptapeptide, or the like, provided that the composition contains at least one dipeptide, tripeptide, or tetrapeptide and at least one pentapeptide, hexapeptide, or heptapeptide. In some embodiments, the compositions comprise a tripeptide and one or more hexapeptides. In some embodiments, the compositions comprise a tripeptide, one or more hexapeptides, and a tetrapeptide. In some embodiments, the tripeptide is tripeptide-1. In some embodiments, the one or more hexapeptide is hexapeptide-12. In some embodiments, the one or more hexapeptide is hexapeptide-11. In some embodiments, the one or more hexapeptide is hexapeptide-38. In some embodiments, the compositions comprise tripeptide-1, hexapeptide-12, hexapeptide-11, and hexapeptide-38. In some embodiments, the tetrapeptide is tetrapeptide-2.

The peptide can be functionalized. For example, the peptide can be functionalized with a fatty acid, e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. Examples include palmitoyl hexapeptide-12 (Pal-VGVAPG), palmitoyl tripeptide-1 (Pal-GHK), myristoyl hexapeptide-12 (Myr-VGVAPG), and myristoyl tripeptide-1 (Myr-GHK). Palmitoyl or myristoyl functionalization can be desirable in certain embodiments as it exhibits enhanced penetration when compared to other fatty acids. In some embodiments, the peptide is functionalized with a chemical group. For example, the peptide is functionalized with acetyl. Examples include acetyl hexapeptide-38 and acetyl tetrapeptide-2. In some instances, the peptide is functionalized with a functional group comprising no more than 14 carbons. In some instances, the peptide is functionalized with a functional group comprising no more than 3, 4, 5, 6, 7, 8, 9, 10, 11, 12, 13, 14, 15, 16, 17, 18, 19, 20, or more than 20 carbons. In some instances, the peptide is non-palmitoylated. Without wishing to be limited to a particular theory, incorporation of the peptide in a liposome, in some embodiments, increases the lipophilicity of a peptide that is functionalized or is not functionalized.

Some embodiments of the methods and compositions provided herein include as a first peptide glycine-histidine-lysine (GHK). GHK is a peptide sequence that is rarely found in the class of proteins in general, but is frequently found in extracellular matrix proteins. The small size of GHK permits it to approach membrane receptors far more easily than larger peptides. Further, its unique, copper-binding structure enhances copper transport into and out of cells and promotes wound healing through several different but related pathways. Due to its strong copper binding structure, GHK can be provided in the form of GHK-Cu (copper-bound GHK form).

In compositions, the tripeptide is typically present in an amount of from about 50 ppm or less to about 100, 200, 300, 400, or 500 ppm or more, e.g., 50 ppm to 150 ppm.

In compositions, the hexapeptide is typically present in an amount of from about 50 ppm or less to about 100, 200, 300, 400, or 500 ppm or more, e.g., 50 ppm to 150 ppm.

In compositions, the tetrapeptide is typically present in an amount of from about 50 ppm or less to about 100, 200, 300, 400, or 500 ppm or more, e.g., 50 ppm to 150 ppm.

The peptides can advantageously be provided in a base for suitable for combining with other components of a liposomal composition. The base can include one or more components such as a thickener/binding agent (e.g., pentaerythrityl tetraisostearate), an emollient/dispersing agent (e.g., caprylic/capric triglyceride), a solvent (e.g., propylene carbonate), and/or a rheology modifier/anti settling agent (e.g., disteardimonium hectorite).

Oleuropein

In some embodiments, polyphenols such as oleuropein may be added to the compositions. Oleuropein is a polyphenol isolated from olive leaves (see e.g. Omar SH. Oleuropein in olive and its pharmacological effects. Sci Pharm 2010; 78(2): 133-54; Al-Rimawi F, Yateem H, Afaneh I. Formulation and evaluation of a moisturizing day cream containing olive leaves extract. International Journal of Development Research 2014; 4(10): 1996-2000; Kontogianni V G, Charisiadis P, Margianni E, Lamari F N, Gerothanassis I P, Tzakos A G. Olive leaf extracts are a natural source of advanced glycation end product inhibitors. Journal of medicinal food 2013; 16(9): 817-22). Oleuropein demonstrates major anti-inflammatory effects by inhibiting lipoxygenase activity and the production of leukotriene. More particularly researchers have demonstrated that oleuropein enhances proteasome activities in vitro more effectively than other known chemical activators, possibly through conformational changes of the proteasome. In this regard, it decreases reactive oxygen species (ROS), reduces the amount of oxidized proteins through increased proteasome-mediated degradation through increased proteasome -mediated degradation and autophagic pathways, and retains proteasome function during replicative senescence.

In some embodiments, compositions as described herein comprise oleuropein. In some embodiments, the oleuropein is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the oleuropein is provided in a range of about 0.001% to about 6%, about 0.002% to about 4%, about 0.01% to about 3%, about 0.02% to about 2%, or about 0.01% to about 0.05% by weight. In some embodiments, the oleuropein is provided at about 0.010% by weight. In some embodiments, the oleuropein is provided at about 0.020% by weight. In some embodiments, the oleuropein is provided at about 0.050% by weight.

Phosphatidylserine

Compositions as described herein, in some embodiments, comprise phosphatidylserine. Exposure of phosphatidylserine from the inner cell membrane of red blood cells can induce phagocytosis of red blood cells. See Chang C F, Goods B A, Askenase M H, et al. Erythrocyte efferocytosis modulates macrophages towards recovery after intracerebral hemorrhage. The Journal of clinical investigation. 2018; 128(2):607-624. Phosphatidylserine can decrease MMP-1, increase procollagen, and stimulate intrinsic production of hyaluronic acid. See Cho S, Kim H H, Lee M J, et al. Phosphatidylserine prevents UV-induced decrease of type I procollagen and increase of MMP-1 in dermal fibroblasts and human skin in vivo. J Lipid Res. 2008; 49(6):1235-1245 and Lee S-H, Yang J-H, Park Y-K, et al. Protective effect and mechanism of phosphatidylserine in UVB-induced human dermal fibroblasts. European Journal of Lipid Science and Technology. 2013; 115(7):783-790.

In some embodiments, phosphatidylserine is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the phosphatidylserine is provided in a range of about 0.25% to about 10%, about 0.5% to about 8%, about 0.75% to about 6%, or about 1% to about 4% by weight. In some embodiments, the phosphatidylserine is provided in a range of about 0.001% to about 6%, about 0.002% to about 4%, about 0.005% to about 0.1%. about 0.01% to about 3%, or about 0.02% to about 2% by weight. In some embodiments, the phosphatidylserine is provided in a range of about 0.005% to about 0.02% by weight. In some embodiments, the phosphatidylserine is provided at about 0.05% by weight. In some embodiments, the phosphatidylserine is provided at about 0.25% by weight. In some embodiments, the phosphatidylserine is provided at about 1% by weight. In some embodiments, the phosphatidylserine is provided at least or about 5, 10, 20, 25, 50, 75, 100, 150, 200, 250, 300, 350, 400, 450, 500, 550, 600, 650, 700, 750, 800, 850, 900, 950, 1000 or more than 1000 microgram per milliliter (ug/mL). In some embodiments, the phosphatidylserine is provided in a range of about 5 to about 1000, about 10 to about 900, about 30 to about 800, about 50 to about 700, about 60 to about 600, or about 100 to about 500 microgram per milliliter (ug/mL).

Candida saitoana

With respect to hydrolyzed Candida saitoana extract, in order to maintain their homeostasis, cells eliminate various accumulated and degraded components. Autophagy, which was recently discovered in skin, stands out today as a powerful mechanism, essential for detoxifying cells and guaranteeing their proper functioning, thereby limiting the senescence. This extract is a purified α-glucan active ingredient, which detoxifies cells by removing altered cell components (oxidized proteins and peroxidized lipids) that saturate them and blocks the accumulation of lipofuscin aggregates, a true marker of aging. See Product monograph: Silab 2013.

In some embodiments, compositions as described herein comprise hydrolyzed Candida saitoana extract. In some embodiments, the hydrolyzed Candida saitoana extract is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hydrolyzed Candida saitoana extract is provided in a range of about 0.25% to about 10%, about 0.5% to about 8%, about 0.75% to about 6%, or about 1% to about 4% by weight. In some embodiments, the hydrolyzed Candida saitoana extract is provided at about 3.0% by weight.

Plantago lanceolata

With respect to Plantago lanceolata, it inhibits micro RNA inhibition of fibroblast function, reversing cellular senescence, thus increasing collagen, laminin, elastin and decreasing MMP-1. See Kovac I, Durkac J, Holly M, et al. Plantago lanceolata L. water extract induces transition of fibroblasts into myofibroblasts and increases tensile strength of healing skin wounds. J Pharm Pharmacol 2015; 67(1): 117-25, and Debacker A, Lavaissiere L, Ringenbach C, Mondon P, Dal Toso R. Controlling MicroRNAs to Fight Skin Senescence. Cosmetics & Toiletries 2016; Feb. 4, 2016: 1-6. Small endogenous noncoding RNAs named microRNA (miRNA) bind to partially complementary sequences of their target messenger RNA (mRNA) and repress or degrade the mRNA, which cause gene inactivation or gene silencing. It appears that collagen I, Collagen IV and elastin are partially controlled by several microRNAs, and when these microRNAs are limited, it helps to boost collagen and elastin synthesis to improve the quality of the dermis. Plantago lanceolata extract was found to reduce the levels of expression of miRNAs controlling the synthesis of collagens and elastin increasing their production and reducing the fibroblast progression toward senescence. Additional in vivo studies demonstrated increased viscoelastic properties with increases in firmness of 30.9% and elasticity of 22.6%, after one month of product application (p<0.01) to the skin.

In some embodiments, compositions as described herein comprise Plantago lanceolata. In some embodiments, the Plantago lanceolata is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Plantago lanceolata is provided in a range of about 0.25% to about 10%, about 0.5% to about 8%, about 0.75% to about 6%, or about 1% to about 4% by weight. In some embodiments, the Plantago lanceolata is provided at about 2.0% by weight.

Arnica montana Extract

Compositions as described herein, in some embodiments, comprise an anti-inflammatory agent. Exemplary anti-inflammatory agents include, but are not limited to, Arnica montana extract. Arnica montana extract includes components such as essential oils, fatty acids, thymol, pseudoguaianolide sesquiterpene lactones, flavanone glycosides, flavonoids, and coumarins. It can exhibit an anti-inflammatory effect. In some instances, Arnica montana extract accelerates healing, reduces bruising potential, modulates inflammation, and stimulates granular tissue and accelerates healing, or combinations thereof. See Rajasingh J, Marzotto M, Bonafini C, et al. Arnica montana Stimulates Extracellular Matrix Gene Expression in a Macrophage Cell Line Differentiated to Wound-Healing Phenotype. PloS one. 2016; 11(11). In some instances, Arnica montana improves bruising by decreasing the inflammation associated with blood products. In some instances, Arnica montana stimulates the function of M2 macrophages and improves wound healing. See Rajasingh J, Marzotto M, Bonafini C, et al. Arnica montana Stimulates Extracellular Matrix Gene Expression in a Macrophage Cell Line Differentiated to Wound-Healing Phenotype. PloS one. 2016; 11(11).

In some embodiments, Arnica montana extract is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 5%, 6%, 7%, 8%, 9%, 10%, or more than 10% by weight (wt. %). In some embodiments, the Arnica montana extract is provided in a range of about 0.25% to about 10%, about 0.5% to about 8%, about 0.75% to about 6%, or about 1% to about 4% by weight. In some embodiments, the Arnica montana extract is provided in a range of about 0.001% to about 6%, about 0.002% to about 4%, about 0.01% to about 3%, about 0.02% to about 2% by weight, or about 0.1% to about 2.5%.

Ledum palustre

Compositions as described herein, in some embodiments, comprise Ledum palustre extract. Ledum palustre is also known as marsh tea, wild rosemary, or labrador tea. Ledum palustre has been used for insect bites, puncture wounds, and cold swellings or bruises. See Kang J Y, Tran K D, Seiff S R, Mack W P, Lee W W. Assessing the Effectiveness of Arnica montana and Rhododendron tomentosum (Ledum palustre) in the Reduction of Ecchymosis and Edema After Oculofacial Surgery: Preliminary Results. Ophthalmic Plast Reconstr Surg. 2017; 33(1):47-52.

In some embodiments, Ledum palustre extract is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the Ledum palustre extract is provided in a range of about 0.25% to about 10%, about 0.5% to about 8%, about 0.75% to about 6%, or about 1% to about 4% by weight. In some embodiments, the Ledum palustre extract is provided in a range of about 0.001% to about 6%, about 0.002% to about 4%, about 0.01% to about 3%, about 0.02% to about 2% by weight, or about 0.1% to about 2.5%. In some embodiments, the Ledum palustre extract is provided at about 0.25%. In some embodiments, the Ledum palustre extract is provided at about 0.5%. In some embodiments, the Ledum palustre extract is provided at about 1.0%.

Leuconostoc/Radish Root Ferment Filtrate

Compositions as described herein, in some embodiments, comprise Leuconostoc/radish root ferment filtrate. In some embodiments, the Leuconostoc/radish root ferment filtrate is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the Leuconostoc/radish root ferment filtrate is provided in a range of about 0.25% to about 10%, about 0.5% to about 8%, about 0.75% to about 6%, or about 1% to about 4% by weight. In some embodiments, the Leuconostoc/radish root ferment filtrate is provided in a range of about 0.001% to about 6%, about 0.002% to about 4%, about 0.01% to about 3%, about 0.02% to about 2% by weight, or about 0.1% to about 2.5%. In some embodiments, the Leuconostoc/radish root ferment filtrate is provided at about 0.25%. In some embodiments, the Leuconostoc/radish root ferment filtrate is provided at about 0.5%. In some embodiments, the Leuconostoc/radish root ferment filtrate is provided at about 1.0%.

Lactoferrin

Compositions as described herein, in some embodiments, comprise a transferrin. In some embodiments, the transferrin is a lactoferrin. In some embodiments, lactoferrin is encapsulated in a liposome. Lactoferrin has wound healing attributes, promotes proliferation of fibroblasts and increases HA secretion. See Saito S, Takayama Y, Mizumachi K, Suzuki C. Lactoferrin promotes hyaluronan synthesis in human dermal fibroblasts. Biotechnology letters. 2011; 33(1):33-39; Takayama Y. Effects of Lactoferrin on Skin Wound Healing. In: Lactoferrin and its Role in Wound Healing. 2012:87-100.

In some instances, the lactoferrin has antimicrobial activity. In some instances, the lactoferrin has antimicrobial activity against bacteria, fungi, yeasts, viruses, parasites, or combinations thereof. Lactoferrin, in some instances, comprises antibiofilm activity. In some instances, lactoferrin interacts with the bacterial surface and destabilizes the microbial membrane. In some instances, lactoferrin chelates iron to disrupt the microbial membrane.

In some embodiments, lactoferrin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the lactoferrin is provided in a range of about 0.005% to about 0.1%, about 0.25% to about 10%, about 0.5% to about 8%, about 0.75% to about 6%, or about 1% to about 4% by weight. In some embodiments, the lactoferrin is provided in a range of about 0.001% to about 6%, about 0.002% to about 4%, about 0.01% to about 2.5%, or about 0.02% to about 2% by weight. In some embodiments, the lactoferrin is provided at about 0.025%. In some embodiments, the lactoferrin is provided at about 0.05%. In some embodiments, the lactoferrin is provided at about 0.10%. In some embodiments, the lactoferrin is provided at least or about 5, 10, 20, 25, 50, 75, 100, 150, 200, 250, 300, 350, 400, 450, 500, 550, 600, 650, 700, 750, 800, 850, 900, 950, 1000 or more than 1000 microgram per milliliter (ug/mL). In some embodiments, the lactoferrin is provided in a range of about 5 to about 1000, about 10 to about 900, about 30 to about 800, about 50 to about 700, about 60 to about 600, or about 100 to about 500 microgram per milliliter (ug/mL).

Dill Extract

Compositions as described herein, in some embodiments, comprise dill extract. The dill extract, in some embodiments, stimulates LOXL reinduction and elastin formation. In some embodiments, the dill extract is Anethum graveolens extract. In some embodiments, the dill extract is Peucedanum graveolens extract.

In some embodiments, the dill extract is provided at least or about 0.01%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the dill extract is provided in a range of about 0.25% to about 10%, about 0.025% to about 4%, about 0.5% to about 8%, about 0.75% to about 6%, or about 1% to about 4% by weight. In some embodiments, the dill extract is provided at about 1.0% by weight.

Hydroxymethoxyphenyl Decanone

Compositions as described herein, in some embodiments, comprise hydroxymethoxyphenyl decanone. In some embodiments, the hydroxymethoxyphenyl decanone is a potent intrinsic hyaluronic acid booster, antioxidant, anti-irritant, or a combination thereof.

In some embodiments, hydroxymethoxyphenyl decanone is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the hydroxymethoxyphenyl decanone is provided in a range of about 0.25% to about 10%, about 0.1% to about 2.5%, about 0.5% to about 8%, about 0.75% to about 6%, or about 1% to about 4% by weight. In some embodiments, the hydroxymethoxyphenyl decanone is provided in a range of about 0.001% to about 6%, about 0.002% to about 4%, about 0.01% to about 3%, or about 0.02% to about 2% by weight.

Tremella fuciformis

Compositions as described herein, in some embodiments, comprise Tremella fuciformis extract. In some embodiments, the Tremella fuciformis extract is derived from an edible mushroom. In some embodiments, Tremella fuciformis extract provides moisture and antioxidant properties.

In some embodiments, Tremella fuciformis extract is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the Tremella fuciformis extract is provided in a range of about 0.25% to about 10%, about 0.1% to about 2.5%, about 0.5% to about 8%, about 0.75% to about 6%, or about 1% to about 4% by weight. In some embodiments, the Tremella fuciformis extract is provided in a range of about 0.001% to about 6%, about 0.002% to about 4%, about 0.01% to about 3%, or about 0.02% to about 2% by weight.

Sodium Hyaluronate Crosspolymer

Compositions as described herein, in some embodiments, comprise sodium hyaluronate crosspolymer. Sodium hyaluronate crosspolymer is a high molecular weight synthetic hyaluronic acid with high water-binding capacity and moisturizing abilities.

In some embodiments, the sodium hyaluronate crosspolymer is provided at least or about 0.0001%, 0.0005%, 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4.0% by weight (wt. %). In some embodiments, the sodium hyaluronate crosspolymer is provided at about 0.5% by weight. In some embodiments, the sodium hyaluronate crosspolymer is provided in a range of about 0.0001% to about 4.0%, about 0.001% to about 4.0%, about 0.01% to about 3.0%, about 0.1% to about 2.5%, or about 0.50% to about 1.5% by weight.

Phytoene and Phytofluene

Compositions as described herein, in some embodiments, comprise phytoene, phytofluene, or combinations thereof. Phytoene and phytofluene are colorless carotenoids derived from saltwater microalgae that modulate Prostaglandin E-2 (PGE-2).

In some embodiments, the phytoene, phytofluene, or combinations thereof is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the phytoene, phytofluene, or combinations thereof is provided in a range of about 0.25% to about 10%, about 0.1% to about 2.5%, about 0.5% to about 8%, about 0.75% to about 6%, or about 1% to about 4% by weight. In some embodiments, the phytoene, phytofluene, or combinations thereof is provided in a range of about 0.001% to about 6%, about 0.002% to about 4%, about 0.01% to about 3%, or about 0.02% to about 2% by weight.

Centella asiatica

In some embodiments, compositions as described herein comprise Centella asiatica. In some embodiments, the Centella asiatica is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Centella asiatica is provided in a range of about 0.25% to about 10%, about 0.5% to about 8%, about 0.75% to about 6%, or about 1% to about 4% by weight. In some embodiments, the Centella asiatica is provided at about 1.0% by weight.

Xylitol

Compositions as described herein, in some embodiments, comprise xylitylglucoside, anhydroxylitol, xylitol, or combinations thereof. Xylitol is a sugar alcohol and comprises anti-biofilm and anti-inflammatory effects. In some embodiments, xylitol and lactoferrin in combination comprise anti-biofilm effects. In some embodiments, xylitol and lactoferrin act synergistically. For example, lactoferrin destabilizes the bacterial membrane and allows xylitol to cross the bacterial membrane to inhibit biofilm development and growth.

In some embodiments, the xylitylglucoside, anhydroxylitol, xylitol, or combinations thereof is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the xylitylglucoside, anhydroxylitol, xylitol, or combinations thereof is provided in a range of about 0.25% to about 10%, about 0.1% to about 2.5%, about 0.5% to about 8%, about 0.75% to about 6%, or about 1% to about 4% by weight. In some embodiments, the xylitylglucoside, anhydroxylitol, xylitol, or combinations thereof is provided in a range of about 0.001% to about 6%, about 0.002% to about 4%, about 0.01% to about 3%, or about 0.02% to about 2% by weight.

Sorbitan Isostearate

Compositions as described herein, in some embodiments, comprise sorbitan isostearate. In some embodiments, the sorbitan isostearate is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the sorbitan isostearate is provided in a range of about 0.25% to about 10%, about 0.1% to about 2.5%, about 0.5% to about 8%, about 0.75% to about 6%, or about 1% to about 4% by weight. In some embodiments, the sorbitan isostearate is provided in a range of about 0.001% to about 6%, about 0.002% to about 4%, about 0.01% to about 3%, or about 0.02% to about 2% by weight. In some embodiments, the sorbitan isostearate is provided at about 0.10% by weight.

Glucose

In some embodiments, compositions as described herein comprise glucose. In some embodiments, the glucose is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the glucose is provided in a range of about 0.25% to about 10%, about 0.1% to about 2.5%, about 0.5% to about 8%, about 0.75% to about 6%, or about 1% to about 4% by weight. In some embodiments, the glucose is provided in a range of about 0.001% to about 6%, about 0.002% to about 4%, about 0.01% to about 3%, or about 0.02% to about 2% by weight. In some embodiments, the glucose is provided at about 0.01% by weight.

Compositions as described herein, in some embodiments, comprise seed oil. In some embodiments, the seed oil is Helianthus annuus (sunflower) seed oil. In some embodiments, the seed oil is provided at least or about 0.001%, 0.003%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the seed oil is provided in a range of about 0.25% to about 10%, about 0.1% to about 2.5%, about 0.5% to about 8%, about 0.75% to about 6%, or about 1% to about 4% by weight. In some embodiments, the seed oil is provided in a range of about 0.001% to about 6%, about 0.002% to about 4%, about 0.01% to about 3%, or about 0.02% to about 2% by weight. In some embodiments, the seed oil is provided at about 0.003% by weight.

Avocado Extract, Shea Butter, Bentonite

In some embodiments, avocado extract is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, avocado extract is provided in a range of about 0.01% to about 5%, about 0.02% to about 4%, 0.05% to about 3%, or about 0.1% to about 2% by weight. In some embodiments, shea butter is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, shea butter is provided in a range of about 0.01% to about 5%, about 0.02% to about 4%, 0.05% to about 3%, or about 0.1% to about 2% by weight. In some embodiments, bentonite is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, bentonite is provided in a range of about 0.01% to about 5%, about 0.02% to about 4%, 0.05% to about 3%, or about 0.1% to about 2% by weight. In some embodiments, avocado extract, shea butter, and bentonite are provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, avocado extract, shea butter, and bentonite are provided in a range of about 0.01% to about 5%, about 0.02% to about 4%, 0.05% to about 3%, about 0.1% to about 2%, or about 0.25% to about 2% by weight. In some embodiments, avocado extract, shea butter, and bentonite are provided at about 0.5% by weight. In some embodiments, avocado extract, shea butter, and bentonite are provided at about 1.0% by weight.

Other Components

Other components can include anti-inflammatory agents, antioxidants, and solubility enhancers. Exemplary anti-irritation agents include, but are not limited to, panthenyl triacetate and naringenin. Panthenyl triacetate and naringenin are natural plant extracts that reduce redness and water loss through the skin. Typical amounts for anti-irritation agents when employed in compositions are from 1% by weight to 4% by weight (wt. %).

Exemplary antioxidant agents include, but are not limited to, Dunaliella salina extract and squalane. Dunaliella salina extract includes components such as beta carotenes. It can exhibit an antioxidant effect. Typical amounts for anti-inflammatory agents when employed in compositions are from 0.1% by weight to 2.5% by weight (wt. %). In some embodiments, the Dunaliella salina extract is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight. In some embodiments, the Dunaliella salina extract is provided in a range of about 0.001% to about 4.0%, about 0.01% to about 3.0%, about 0.1% to about 2.5%, or about 0.50% to about 1.5%. In some embodiments, the squalane is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight. In some embodiments, the squalane is provided in a range of about 0.001% to about 4.0%, about 0.01% to about 3.0%, about 0.1% to about 2.5%, or about 0.50% to about 1.5%. In some embodiments, the Dunaliella salina extract and the squalane is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight. In some embodiments, the Dunaliella salina and the squalane extract is provided in a range of about 0.001% to about 4.0%, about 0.01% to about 3.0%, about 0.1% to about 2.5%, or about 0.50% to about 1.5%.

In some embodiments, the composition comprises a siloxane polymer. In some embodiments, the siloxane polymer is caprylyl methicone. In some embodiments, caprylyl methicone is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4.0% by weight (wt. %). In some embodiments, the caprylyl methicone is provided at about 0.5% by weight. In some embodiments, the caprylyl methicone is provided in a range of about 0.001% to about 4.0%, about 0.01% to about 3.0%, about 0.1% to about 2.5%, or about 0.50% to about 1.5% by weight. In some embodiments, the caprylyl methicone is provided at about 0.25% by weight. In some embodiments, the caprylyl methicone is provided at about 1% by weight.

In some embodiments, compositions as described herein comprise Euglena gracilis extract, aqua, caffeine, Glaucium flavum leaf extract, or combinations thereof. In some embodiments, compositions as described herein comprising Euglena gracilis extract, aqua, caffeine, and Glaucium flavum leaf extract are each provided or provided together at least or about 0.001%, 0.002%, 0.005%, 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Euglena gracilis extract, aqua, caffeine, and Glaucium flavum leaf extract are each provided or provided together in a range of about 0.001% to about 6%, about 0.002% to about 4%, about 0.01% to about 3%, or about 0.02% to about 2% by weight. In some embodiments, the Euglena gracilis extract, aqua, caffeine, and Glaucium flavum leaf extract are each provided or provided together at about 0.20% by weight.

In some embodiments, the caffeine is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the caffeine is provided in a range of about 0.001% to about 6%, about 0.002% to about 4%, about 0.01% to about 3%, or about 0.02% to about 2%. In some embodiments, caffeine is provided with sodium salicylate, lecithin, silica, or combinations thereof. In some embodiments, the sodium salicylate, lecithin, or silica are each provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.)

In some embodiments, compositions as described herein comprise polyholosides. In some embodiments, the polyholosides are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the polyholosides are provided in a range of about 0.25% to about 10%, about 0.5% to about 8%, about 0.75% to about 6%, about 1% to about 4%, or about 2.5% to about 10% by weight. In some embodiments, the polyholosides are provided at about 5.0% by weight.

Compositions as described herein, in some embodiments, comprise ceramide NP. In some embodiments, the ceramide NP is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the ceramide NP is provided in a range of about 0.001% to about 6%, about 0.002% to about 4%, about 0.01% to about 3%, about 0.02% to about 2%, or about 0.50% to about 0.20% by weight. In some embodiments, the ceramide NP is provided at about 0.05% by weight. In some embodiments, the ceramide NP is provided at about 0.10% by weight. In some embodiments, the ceramide NP is provided at about 0.20% by weight.

Bentonite clays can be employed in conjunction with the peptides to provide impart penetration and adsorption properties to the compositions, and can aid in stabilizing emulsions. Other clays, such as hectorite and magnesium aluminum silicate can also be employed. Bentonite or other clays can be modified to yield an organic modified clay compound. Salts (e.g., quaternary ammonium salts) of fatty acids (e.g., hydrogenated fatty acids) can be reacted with hectorite or other clays. As provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δn, where n indicates the lower numbered carbon of each pair of double-bonded carbon atoms. A shorthand notation specifying total # carbons : # double bonds, Δ_{double bond positions} can be employed. For example, 20:4Δ_{5,8,11,14} refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-Δ9-octadecenoate) is a monounsaturated fatty acid, linolenate (all-cis-Δ9,12,15-octadecatrienoate) is a polyunsaturated fatty acid. Fatty acids suitable for use can comprise from 5 to 30 carbon atoms, e.g., 5, 6, 7, 8, 9, 10, 11, 12, 13, 14, 15, 16, 17, 18, 19, 20, 21, 22, 23, 24, 25, 26, 27, 28, 29, or 30 carbon atoms. The fatty acid can be fully saturated, or can include as many double bonds as are feasible for the chain length. Fatty acids suitable for functionalizing hectorite or other clays include palmitic acid and stearic acid. Dialkyl quaternary cationic modifiers include dipalmoyldimonium chloride and distearyldimonium chloride. Amidoamine quaternary cationic modifiers include palmitamidopropyltrimonium chloride cetearyl alcohol and palmitamidopropyltrimonium chloride.

In some embodiments, the peptides can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for compositions include, without limitation, monoglycerides, diglycerides, sulfatides, lysolecithin, phospholipids, saponin, bile acids, and the like. In some embodiments, compositions described herein comprise, phosphatidylserine, phospholipids, tocopherol, ascorbyl palmitate, or combinations thereof. In some embodiments, phosphatidylserine, phospholipids, tocopherol, ascorbyl palmitate, or combinations thereof is provided at 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the phosphatidylserine, phospholipids, tocopherol, ascorbyl palmitate, or combinations thereof is provided in a range of about 0.25% to about 10%, about 0.5% to about 8%, about 0.75% to about 6%, or about 1% to about 4% by weight. In some embodiments, the phosphatidylserine, phospholipids, tocopherol, ascorbyl palmitate, or combinations thereof is provided in a range of about 0.001% to about 6%, about 0.002% to about 4%, about 0.01% to about 3%, or about 0.02% to about 5% by weight. In some embodiments, the additive is betaine. Betaine, in some embodiments, is provided in a range of about 0.001% to about 6%, about 0.002% to about 4%, about 0.01% to about 3%, or about 0.02% to about 5% by weight. In some embodiments, the compositions as described herein comprise caprylyl glycol. In some embodiments, the caprylyl glycol provided in a range of about 0.001% to about 6%, about 0.002% to about 4%, about 0.01% to about 3%, or about 0.02% to about 5% by weight. In some embodiments, the compositions as described herein comprise caprylhydroxamic acid. In some embodiments, the caprylhydroxamic acid provided in a range of about 0.001% to about 6%, about 0.002% to about 4%, about 0.01% to about 3%, or about 0.02% to about 5% by weight. The presence of such additional components can influence the physical state, solubility, stability, rate of release, rate of clearance, and penetration of active ingredients.

The compositions for topical administration comprise the peptide compositions as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the peptides into the skin. An alternative to increasing molecular weight (as with silicone gums) or adding filler (as with silicone compounds) is to partially crosslink siloxane polymers and disperse this material in an appropriate silicone carrier fluid. The resulting dimethicone crosspolymers (also known as silicone elastomers in the personal care industry) differ from basic polydimethylsiloxane (PDMS) because of the cross-linking between the linear polymers. These materials can be employed in peptide compositions, and also offer benefits in scar treatment, periwound protection and enzyme delivery. In skin care applications, the aesthetics of silicone elastomers (including those with functional groups) and their ability to absorb various oils (e.g., with a dimethicone/vinyl dimethicone crosspolymer such as Dow Corning® 9506 Elastomer Powder) are two of the elastomer's desirable properties. Silicone elastomers have a skin feel different from any of the silicone fluids, described as “smooth,” “velvety,” and “powdery.” It can be modified by controlling the amount of liquid phase in the formula, and therefore the degree of swelling. Due to their film-forming properties, dimethicone crosspolymers can be used as delivery systems for active ingredients such as the peptides described herein, or other composition components such as oil-soluble vitamins and sunscreens. Sunscreens such as octyl methoxycinnamate can be more efficiently delivered from a composition containing a silicone elastomer, producing a higher sun protection factor (SPF). Silicone elastomer blends can be used to enhance SPF in oil-in-water compositions containing organic sunscreens. For example, in testing conducted regarding SPF, the addition of 4% silicone elastomer blend to a sun care composition containing organic sunscreens increased the SPF from 5.7 to 18. This property of the silicone elastomer allows the effectiveness of sunscreen agents in a composition to be maximized while reducing the amount needed to achieve a desired SPF. As a result, composition costs can be reduced along with potential irritation caused by sunscreen actives. Accordingly, a higher SPF can be achieved with the same amount of UV absorber, resulting in enhanced performance with no added composition cost. Silicone elastomers can be produced from linear silicone polymers by a variety of crosslinking reactions, e.g., by a hydrosilylation reaction in which a vinyl group reacts with a silicon hydride. The general process involves linear silicone polymers with reactive sites along the polymer chain reacting with a cross-linker. The dimethicone crosspolymer can be produced either as a gel made of a suspension of elastomer particles swollen in a carrier fluid (e.g., a mixture of high molecular weight silicone elastomer in cyclopentasiloxane such as Dow Corning® 9040 Silicone Elastomer Blend), or as a spray-dried powder (a dimethicone/vinyl dimethicone crosspolymer such as Dow Corning® 9506 Elastomer Powder). The gel form having desirable attributes is cyclomethicone, but low viscosity dimethicones and organic fluids can also be used. Examples of dimethicone crosspolymers in the suspension or gel form are high molecular weight silicone elastomer (12%) in decamethylcyclopentasiloxane (e.g., Dow Corning® ST-Elastomer 10) and a mixture of high molecular weight silicone elastomer in cyclopentasiloxane (e.g., Dow Corning® 9040 Silicone Elastomer Blend), which typically have an elastomer content ranging from 10 to 20% by weight.

The pharmaceutical excipients used in the topical preparations of the peptide compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic liposomal composition include water; ethyl alcohol; isopropyl alcohol; mixtures of water and ethyl and/or isopropyl alcohols; glycerin; ethylene, propylene or butylene glycols; DMSO; pentylene glycol; and mixtures thereof. In some embodiments, glycerin is provided at least or about 1%, 2%, 3%, 4%, 5%, 6%, 7%, 8%, 9%, 10%, 11%, 12%, or more than 12% by weight (wt. %). In some embodiments, glycerin is provided at least or about 7%. In some embodiments, glycerin is provided in a range of about 1% to about 12%, about 2% to about 11%, or about 3% to about 10% by weight. In some embodiments, butylene glycol is provided at least or about 0.0025%, 0.005%, 0.075%, 0.01%, 0.025%, 0.05%, 0.75%, 1%, 2%, 3%, 4%, 5%, 6%, 7%, 8%, 9%, 10%, 11%, 12%, or more than 12% by weight. In some embodiments, butylene glycol is provided in a range of about 0.01% to about 10%, about 0.025% to about 5%, or about 0.05% to about 1.25% by weight. In some embodiments, pentylene glycol is provided at least or about 0.0025%, 0.005%, 0.075%, 0.01%, 0.025%, 0.05%, 0.75%, 1%, 2%, 3%, 4%, 5%, 6%, 7%, 8%, 9%, 10%, 11%, 12%, or more than 12% by weight. In some embodiments, pentylene glycol is provided in a range of about 0.01% to about 10%, about 0.025% to about 5%, or about 0.05% to about 1.25% by weight. Suitable solvents for hydrophobic compositions include mineral oils, vegetable oils, and silicone oils. If desired, the peptide compositions as described herein may be dissolved or dispersed in a hydrophobic oil phase, and the oil phase may then be emulsified in an aqueous phase comprising water, alone or in combination with lower alcohols, glycerin, and/or glycols. In some embodiments, an anhydrous composition is applied as the presence of water can result in stinging upon administration to skin tissues subject to laser treatment, chemical peel, dermabrasion, or the like. Anhydrous compositions may also act to prevent the development of water-based irritant contact dermatitis in damaged or sensitive skin, which may produce rashes and skin irritation that may retard wound healing and improvement in skin quality. Tsai, T. F., Maibach, H. I. How irritant is water? An overview. Contact Dermatitis 41(6) (1999): 311-314 (describing contact dermatitis caused by water as an irritant). However, in certain embodiments it may be acceptable to provide water based compositions, or to permit a limited amount of water to be present. For example, water may be present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the compositions as described herein can be advantageously employed where it is desirable to minimize osmotic shock.

Compositions as described herein may comprise varying amounts of solvent. In some embodiments, the solvent is water. In some embodiments, the solvent is at least or about 10%, 15%, 20%, 25%, 30%, 35%, 40%, 45%, 50%, 55%, 60%, 65%, 70%, 75%, 80%, 85%, 90%, 95%, or more than 95% by weight (wt. %). In some embodiments, the solvent is in a range of about 10% to about 95%, about 20% to about 90%, about 30% to about 85%, about 40% to about 80%, or about 50% to about 75% by weight.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

The viscosity of the compositions as described herein, in some embodiments, are in a range of about 8,000 centipoise (cps) to about 30,000 cps. In some embodiments, the viscosity is at least or about 4,000; 5,000; 6,000; 7,000; 8,000; 9,000; 10,000; 11,000; 12,000; 13,000; 14,000; 15,000; 16,000; 17,000; 18,000; 19,000; 20,000; 21,000; 22,000; 23,000; 24,000; 25,000; 26,000; 27,000; 28,000;29,000; 30,000; 31,000; 32,000; 33,000; 34,000, 35,000; 36,000; 37,000; 38,000; 39,000; 40,000; or more than 40,000 cps. In some embodiments, the composition comprises a viscosity in a range of about 4,000 to about 40,000, about 6,000 to about 38,000, about 8,000 to about 36,000, about 10,000 to about 34,000 cps, about 12,000 to about 32,000 cps, or about 14,000 to about 30,000 cps.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids. In some embodiments, the emollient is caprylic/capric triglyceride.

In some embodiments, the emollient is provided at least or about 0.0025%, 0.005%, 0.075%, 0.01%, 0.025%, 0.05%, 0.75%, 1%, 2%, 3%, 4%, 5%, 6%, 7%, 8%, 9%, 10%, 11%, 12%, or more than 12% by weight. In some embodiments, the emollient is provided in a range of about 0.01% to about 10%, about 0.01% to about 2.5%, about 0.025% to about 5%, or about 0.05% to about 1.25% by weight. In some embodiments, the caprylic/capric triglyceride is provided at least or about 0.0025%, 0.005%, 0.075%, 0.01%, 0.025%, 0.05%, 0.75%, 1%, 2%, 3%, 4%, 5%, 6%, 7%, 8%, 9%, 10%, 11%, 12%, or more than 12% by weight. In some embodiments, the caprylic/capric triglyceride is provided in a range of about 0.01% to about 10%, about 0.01% to about 2.5%, about 0.025% to about 5%, or about 0.05% to about 1.25% by weight.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the compositions include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the compositions. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in compositions include anionic, cationic, nonionic, and zwitterionic surfactants. Preferred ionic emulsifiers include phospholipids, such as lecithin and derivatives.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in compositions include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the composition. It is generally observed that the anhydrous compositions of the embodiments exhibit satisfactory stability, such that a preservative can be omitted from the composition.

Suitable chelating agents for use in compositions include ethylene diamine tetraacetic acid, alkali metal salts thereof alkaline earth metal salts thereof, ammonium salts thereof, and tetraalkyl ammonium salts thereof. In some embodiments, the chelating agent is disodium ethylenediaminetetraacetic acid (EDTA). In some embodiments, the disodium EDTA is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the disodium EDTA is provided in a range of about 0.25% to about 10%, about 0.1% to about 2.5%, about 0.5% to about 8%, about 0.75% to about 6%, or about 1% to about 4% by weight. In some embodiments, the disodium EDTA is provided in a range of about 0.001% to about 6%, about 0.002% to about 4%, about 0.01% to about 3%, or about 0.02% to about 2% by weight.

The carrier preferably has a pH of between about 4.0 and 10.0, more preferably between about 4.8 and about 7.8, more preferably between about 5.0 to about 6.5. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The peptide compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the composition, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

In some embodiments, compositions comprise phenoxyethanol, ethylhexylglycerin, or combinations thereof. In some embodiments, phenoxyethanol is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, phenoxyethanol is provided in a range of about 0.25% to about 10%, about 0.5% to about 8%, about 0.75% to about 6%, or about 1% to about 4% by weight. In some embodiments, ethylhexylglycerin is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, ethylhexylglycerin is provided in a range of about 0.25% to about 10%, about 0.5% to about 8%, about 0.75% to about 6%, or about 1% to about 4% by weight. In some embodiments, phenoxyethanol and ethylhexylglycerin are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, phenoxyethanol and ethylhexylglycerin are provided in a range of about 0.25% to about 10%, about 0.1% to about 4%, about 0.5% to about 8%, about 0.75% to about 6%, or about 1% to about 4% by weight.

In some embodiments, compositions comprise polyacrylate-13, polyisobutene, polysorbate 20, or combinations thereof. In some embodiments, polyacrylate-13 is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, polyacrylate-13 is provided in a range of about 0.25% to about 10%, about 0.5% to about 8%, about 0.75% to about 6%, or about 1% to about 4% by weight. In some embodiments, polyisobutene is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, polyisobutene is provided in a range of about 0.25% to about 10%, about 0.5% to about 8%, about 0.75% to about 6%, or about 1% to about 4% by weight. In some embodiments, polyacrylate-13 is provided in a range of about 0.25% to about 10%, about 0.5% to about 8%, about 0.75% to about 6%, or about 1% to about 4% by weight. In some embodiments, polysorbate 20 is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, polysorbate 20 is provided in a range of about 0.25% to about 10%, about 0.5% to about 8%, about 0.75% to about 6%, or about 1% to about 4% by weight. In some embodiments, polyacrylate-13, polyisobutene, and polysorbate 20 are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, polyacrylate-13, polyisobutene, and polysorbate 20 are provided in a range of about 0.25% to about 10%, about 0.1% to about 4%, about 0.5% to about 8%, about 0.75% to about 6%, or about 1% to about 4% by weight (wt. %).

In some embodiments, compositions as described herein comprise potassium sorbate. In some embodiments, the potassium sorbate is provided at least or about 0.001%, 0.00175%, 0.0025%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the potassium sorbate is provided in a range of about 0.001% to about 6%, about 0.002% to about 4%, about 0.01% to about 3%, or about 0.02% to about 2% by weight.

In some embodiments, the liposomes comprise propanediol, lecithin, or a combination thereof. In some embodiments, the propanediol is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol is provided in a range of about 0.001% to about 6%, about 0.002% to about 4%, about 0.01% to about 3%, or about 0.02% to about 2% by weight. In some embodiments, the lecithin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the lecithin is provided in a range of about 0.001% to about 6%, about 0.002% to about 4%, about 0.01% to about 3%, or about 0.02% to about 2% by weight. In some embodiments, the liposomes comprise propanediol and lecithin. In some embodiments, the propanediol and lecithin are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol and lecithin are provided in a range of about 0.001% to about 6%, about 0.002% to about 4%, about 0.01% to about 3%, or about 0.02% to about 2% by weight. In some embodiments, the propanediol and lecithin are provided at about 0.90% by weight.

The topical composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting composition contains micelles, i.e., spherical oil droplets

Penetration Enhancers

Fatty acids and alcohols can be employed to enhance penetration of the peptides, and to provide a silky feel to compositions, e.g., methanoic acid, ethanoic acid, propanoic acid, butanoic acid, isobutyric acid, pentanoic acid, hexanoic acid, heptanoic acid, octanoic acid, nonanoic acid, decanoic acid, myristoleic acid, isovaleric acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, medium chain fatty acids, e.g., C₆₋₁₂ fatty acids, or the like. Typical amounts when employed in compositions are from 1% by weight to 4% by weight.

Antimicrobial Efficacy

Described herein, in some embodiments, are chemically and physically stable compositions at physiological pH. In some embodiments, the compositions are sterile and safe for human administration. In some embodiments, the compositions comply with or pass the required antimicrobial efficacy tests such as the Antimicrobial Effectiveness Test. In some embodiments, the compositions result in complete or substantially complete eradication of bacteria, yeast, mold, or combinations thereof.

Liposomes

Described herein are liposomal compositions for improved distribution, efficacy, bioavailability, and/or activity. Liposomal compositions may improve distribution, efficacy, bioavailability, and/or activity of the active ingredient by improving delivery and tissue (e.g. skin) penetration. In some instances, improved delivery and skin penetration result from the active ingredient being incorporated (e.g. encapsulated) in a liposome. In some instances, the active ingredient is a peptide that is encapsulated in a liposome.

Liposomal compositions as described herein may comprise a peptide encapsulated in a liposome. In some embodiments, the peptide is tripeptide-1. In some embodiments, the peptide is hexapeptide-12. In some embodiments, the peptide is hexapeptide-11 In some embodiments, the peptide is hexapeptide-38. In some embodiments, the peptide is tetrapeptide-2. In some embodiments, the peptide is functionalized with a palmitoyl group. In some embodiments, the peptide is functionalized with an acetyl group. For example, the peptide is acetyl hexapeptide-38.

Liposomal compositions as described herein may comprise various ingredients encapsulated in a liposome. In some embodiments, the ingredient is lactoferrin. In some embodiments, the ingredient is phosphatidylserine. In some embodiments, the ingredient is Ledum palustre extract. In some embodiments, the ingredient is Arnica montana extract. In some embodiments, the ingredient is sodium hyaluronate. In some embodiments, the ingredient is larger than 50 kDa.

Lecithin and other phospholipids may be used to prepare liposomes containing the peptide compositions as described herein. In some embodiments, liposomes are used to prepare one or more peptides. In some embodiments, the peptide is functionalized with an acetyl group. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the peptide compositions as described herein.

The phospholipids used to prepare the liposomal compositions described herein may comprise a transition phase temperature of about 10° C. to about 25° C. In some instances, the phospholipids comprise a transition phase temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the phospholipids comprise a transition phase temperature in a range of about 10° C. to about 40° C., about 12° C. to about 36° C., about 14° C. to about 32° C., about 16° C. to about 20° C., or about 21° C. to about 25° C.

The topical composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting composition contains micelles, i.e., spherical oil droplets.

The liposomal composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Described herein, in some embodiments, are methods for preparing a composition comprising a peptide encapsulated in a liposome, comprising: combining the peptide and a solvent to form a mixture; and contacting the mixture with an aqueous solution comprising liposomes. In some instances, the contacting occurs at a temperature between about 10° C. and about 25° C. In some instances, the contacting occurs at a temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the contacting occurs at a temperature in a range of about 10° C. to about 40° C., about 12° C. to about 36° C., about 14° C. to about 32° C., about 16° C. to about 20° C., or about 21° C. to about 25° C.

Methods for preparing a composition comprising a peptide encapsulated in a liposome may comprise use of a solvent. In some instances, the solvent is water. In some instances, the solvent is an organic solvent. Exemplary organic solvents include, but are not limited to, petroleum ether, cyclohexane, toluene, carbon tetrachloride, dichloromethane, chloroform, diethyl ether, diisopropyl ether, ethyl acetate, butanol, n-propanol, ethanol, methanol, polyethylene glycol, propylene glycol, and pyridine. In some instances, the solvent is a glycol. In some instances, the solvent is butylene glycol. In some instances, the solvent is caprylyl glycol. In some instances, the solvent is propanediol (propylene glycol).

The solvent may be used at various percentages. In some instances, the solvent is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10%. The solvent may be propanediol, butylene glycol, or caprylyl glycol.

Methods as described herein, in some embodiments, comprises combining the peptide and a solvent to form a mixture; and contacting the mixture with an aqueous solution comprising liposomes, wherein the aqueous solution comprises a percentage of water and a percentage of liposomes. In some instances, the aqueous solution comprises at least or about 20%, 30%, 40%, 50%, 60%, 70%, 80%, 90%, or more than 90% water. In some instances, the aqueous solution comprises water in a range of about 10% to about 95%, about 20% to about 90%, about 30% to about 85%, about 40% to about 80%, or about 50% to about 60%. In some instances, the aqueous solution comprises at least or about 20%, 30%, 40%, 50%, 60%, or more than 60% liposomes. In some instances, the aqueous solution comprises liposomes in a range of about 10% to about 80%, about 20% to about 70%, or about 30% to about 60%. A ratio of liposomes to water may be in a range of about 1:9 to about 3:7. In some instances, the ratio of liposomes to water may be at least or about 1:10, 1:9, 1:8, 1:7, 1:6, 1:5, 1:4, 1:3, or 1:2.

Methods for generation of liposomal compositions as described herein may result in an entrapment efficacy of no more than 100%. In some instances, the entrapment efficacy is no more than 50%, 60%, 70%, 80%, 90%, 95%, 99%, or 99.5%.

Described herein are liposomal compositions, wherein the peptide comprises a percentage of the composition. In some embodiments, the peptide is provided at least or about 0.0001%, 0.0005%, 0.00055%, 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% of the composition. In some embodiments, the peptide is provided at least or about 10%, 11%, 12%, 13%, 14%, 15%, 16%, 17%, 18%, 19%, 20%, 22%, 24%, 26%, 28%, 30% or more than 30% of the composition. In some embodiments, the peptide is provided in a range of about 0.001% to about 6%, about 0.002% to about 4%, about 0.01% to about 5%, or about 0.02% to about 2% by weight. In some embodiments, the peptide is provided at about 0.03% of the composition.

Described herein are liposomal compositions, wherein the liposomes comprise a percentage of the composition. In some embodiments, the liposomes are provided at least or about 10%, 11%, 12%, 13%, 14%, 15%, 16%, 17%, 18%, 19%, 20%, 22%, 24%, 26%, 28%, 30% or more than 30% of the composition. In some embodiments, the liposomes are provided in a range of about 5% to about 90%, about 10% to about 80%, about 20% to about 70%, about 30% to about 60%, about 10% to about 30%, or about 20% to about 40%. In some embodiments, the liposomes are provided at about 30%. In some embodiments, the liposomes are provided at 27%.

Liposomal compositions as described herein, in some embodiments, comprise an average particle size of at most 220 nanometers (nm). In some instances, the average particle size is at most 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is about 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is in a range of about 50 nm to about 500 nm, about 100 nm to about 400 nm, about 150 nm to about 220 nm, about 180 nm to about 220 nm, or about 190 nm to about 210 nm.

In some instances, the liposomal compositions comprise an active agent that has a molecular weight of no more than about 600 Daltons (Da). In some instances, the active agent has a molecular weight of at least or about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, or more than 1000 Daltons (Da). In some instances, the active agent has a molecular weight of at least or about 1000, 1100, 1200, 1300, 1400, 1500, 1600, 1700, 1800, 1900, 2000, 2100, 2200, 2300, 2400, 2500, 2600, 2700, 2800, 2900, 3000, 4000, 5000, 6000, or more than 6000 Daltons (Da). In some instances, the active agent has a molecular weight in a range of about 50 to about 1000, about 100 to about 900, about 200 to about 800, about 300 to about 700, or about 400 to about 600 Daltons (Da). In some instances, the active agent is a peptide. In some instances, the active agent is a peptide encapsulated in a liposome.

A polydispersity index (PdI) of a liposomal composition as described herein, in some embodiments, is in a range of 0 to about 0.2. In some instances, the polydispersity index is about 0.01, 0.025, 0.05, 0.1, 0.25, 0.3, 0.35, 0.4, 0.45, 0.5, 0.55, 0.6, 0.65, 0.7, 0.75, or 0.8. In some instances, the polydispersity index is in a range of about 0.01 to about 0.8, about 0.025 to about 0.75, about 0.05 to about 0.6, or about 0.1 to about 0.3.

In some instances, an intercept of a liposomal composition as described herein is in a range of about 0.85 to about 0.95. In some instances, the intercept is the amplitude. In some instances, the intercept is at least or about 0.65, 0.70, 0.75, 0.80, 0.85, 0.90, or 0.95.

In some embodiments, the liposomes comprise propanediol, lecithin, or a combination thereof. In some embodiments, the propanediol is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol is provided in a range of about 0.001% to about 6%, about 0.002% to about 4%, about 0.01% to about 3%, or about 0.02% to about 2% by weight. In some embodiments, the lecithin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the lecithin is provided in a range of about 0.001% to about 6%, about 0.002% to about 4%, about 0.01% to about 3%, or about 0.02% to about 2% by weight. In some embodiments, the liposomes comprise propanediol and lecithin. In some embodiments, the propanediol and lecithin are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol and lecithin are provided in a range of about 0.001% to about 6%, about 0.002% to about 4%, about 0.01% to about 3%, or about 0.02% to about 2% by weight. In some embodiments, the propanediol and lecithin are provided at about 0.90% by weight

Described herein are liposomal compositions comprising improved distribution, efficacy, bioavailability, and/or activity. The liposomal compositions may comprise improved distribution, efficacy, bioavailability, and/or activity as compared to compositions not comprising liposomes. In some instances, the distribution is improved by at least or about 0.5×, 1.0×, 1.5×, 2.0×, 2.5×, 3.0×, 4.0×, 4.5×, 5×, or more than 5× as compared to compositions not comprising liposomes. In some instances, the efficacy is improved by at least or about 0.5×, 1.0×, 1.5×, 2.0×, 2.5×, 3.0×, 4.0×, 4.5×, 5×, or more than 5× as compared to compositions not comprising liposomes. In some instances, the bioavailability is improved by at least or about 0.5×, 1.0×, 1.5×, 2.0×, 2.5×, 3.0×, 4.0×, 4.5×, 5×, or more than 5× as compared to compositions not comprising liposomes. In some instances, the activity is improved by at least or about 0.5×, 1.0×, 1.5×, 2.0×, 2.5×, 3.0×, 4.0×, 4.5×, 5×, or more than 5× as compared to compositions not comprising liposomes. The distribution, efficacy, bioavailability, and/or activity may be improved by at least or about 10%, 15%, 20%, 25%, 30%, 35%, 40%, 45%, 50%, 55%, 60%, 65%, 70%, 75%, 80%, 85%, 90%, or more than 90% as compared to compositions not comprising liposomes.

Liposomal compositions and methods as described herein, in some embodiments, are topical compositions. In some instances, the liposomal compositions are oil free. In some instances, the liposomal compositions are preservative free. In some embodiments, the liposomal formulation is an aqueous formulation. In some embodiments, the liposomal formulation is an anhydrous formulation. In some instances, the liposomal composition comprises a pH in a range of about 5 to about 8. In some instances, the liposomal composition comprises a pH of at least or about 2, 3, 4, 5, 6, 7, 8, 9, or 10.

Methods of Use

Described herein are compositions and methods for restoring dermal integrity and vessel support, recycling the ECM, increasing collagen, elastin and glycosaminoglycans (GAGS) such as hyaluronic acid (HA), replacing waste products with new matrix components, and combinations thereof. Compositions and methods described herein can be used for treating dermatoporosis or for promoting restoration of aging skin.

Compositions and methods as described herein can promote restoration of aging skin. In some instances, the aging skin is thinner, less elastic, and more prone to bruising. In some instances, the aging skin is characterized by atrophy or alteration in collagen, elastin, and components of the extracellular matrix. This atrophy results in a lack of support for aged vessels resulting in leakage of blood products and in some cases rupture of the vessels. Compositions and methods as described herein can promote restoration of aging skin by restoring dermal integrity and vessel support, recycling the ECM, increasing collagen, elastin and GAGs, replacing waste products with new matrix components, and combinations thereof.

In some embodiments, the aging skin is more prone to bruising. In some embodiments, the subject more prone to bruising is an elderly individual having capillary fragility. In some instances, the aging skin is subject to photodamage. In some instances, the aging skin is exposed to solar radiation. In some instances, the aging skin is exposed to actinic damage.

Described herein are methods and compositions for improving various types of bruises in an elderly individual. In some embodiments, the elderly individual has chronic skin fragility. In some embodiments, the elderly individual has dermatoporosis. In some embodiments, the elderly individual has a nonpalpable (macular) purpura, a palpable (papular) purpura, a nonthrombocytopenic purpura, a thrombocytopenic purpura, or a senile purpura. In some embodiments, the elderly individual has a senile purpura.

Compositions and methods as described herein may be used for treating the various stages of dermatoporosis. In some instances, the compositions and methods as described herein are used to treat stage I of dermatoporosis characterized by skin atrophy, senile purpura and pseudocicatrices. In some instances, the compositions and methods as described herein are used to treat stage Ha of dermatoporosis characterized by small superficial lacerations (<3 cm) due to skin fragility. In some instances, the compositions and methods as described herein are used to treat stage IIb of dermatoporosis characterized by larger lacerations (>3 cm). In some instances, the compositions and methods as described herein are used to treat stage IIIa of dermatoporosis characterized by superficial hematomas. In some instances, the compositions and methods as described herein are used to treat stage IIIb of dermatoporosis characterized by deep dissecting haematomas (DDH) without skin necrosis. In some instances, the compositions and methods as described herein are used to treat stage IV of dermatoporosis characterized by large areas of skin necrosis with potential lethal complications.

Compositions as described herein, in some embodiments, improve morphological features of aging skin. In some instances, the compositions promote restoration of aging skin. In some instances, the morphological features comprise aging spots. In some instances, the morphological features comprise white pseudoscars. In some instances, the morphological features comprise uneven skin tone. In some instances, the morphological feature comprises wrinkles. In some instances, the morphological feature comprises purpura. In some instances, the morphological features comprise a bruise.

In some embodiments, the compositions as described herein improve the morphological features by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the compositions as described herein improve the morphological features by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, the compositions improve bruising by accelerating resolution of the bruise. For example, the compositions accelerate the transition of blue coloration to red coloration of the bruise. In some embodiments, improved appearance of the bruise comprises reduced size of the bruise. In some embodiments, improved appearance of the bruise comprises reduced discoloration of the skin. In some embodiments, improved appearance of the bruise comprises reduced swelling. In some embodiments, the compositions as described herein improve bruising by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the compositions as described herein improve bruising by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×.

Compositions as described herein may be used with various treatment regimens. In some instances, the topical compositions described herein are administered once per day, twice per day, three times per day or more. In some instances, the topical compositions described herein are administered twice per day. The topical compositions described herein, in some embodiments, are administered daily, every day, every alternate day, five days a week, once a week, every other week, two weeks per month, three weeks per month, once a month, twice a month, three times per month, or more. In some embodiments, the topical compositions described herein are administered twice daily, e.g., morning and evening. In some embodiments, the topical compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, the topical compositions described herein are administered twice daily for at least or about 1 week, 2 weeks, 3 weeks, 1 month, 2 months, 3 months, 4 months, 5 months, 6 months, or more. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more.

In some instances, the compositions described herein are administered up to 1 day, up to 2 days, up to 3 days, up to 5 days, up to 6 days, up to 1 week, up to 2 weeks, up to 3 weeks, or more than 3 weeks prior to appearance of morphological features associated with aging. In some instances, the compositions described herein are administered immediately prior to appearance of morphological features associated with aging including up to 1 hour, up to 2 hours, up to 3 hours, up to 5 hours, up to 6 hours, up to 7 hours, up to 8 hours, up to 12 hours, up to 16 hours, up to 20 hours, or up to 24 hours prior to the procedure or trauma. Sometimes the compositions described herein are administered singly, or over a time course, such as daily, multiple times weekly, weekly, biweekly, monthly or less frequently prior to appearance of morphological features associated with aging. In some instances, the compositions described herein are administered singly, or over a time course, such as daily, multiple times weekly, weekly, biweekly, monthly or more frequently prior to appearance of morphological features associated with aging. In some embodiments, the compositions are topical compositions. In some instances, the topical compositions are administered twice daily for at least or about 1 week, 2 weeks, 3 weeks, 1 month, 2 months, 3 months, 4 months, 5 months, 6 months, or more prior to appearance of morphological features associated with aging. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more prior to appearance of morphological features associated with aging.

In some instances, the compositions described herein are administered up to 1 day, up to 2 days, up to 3 days, up to 5 days, up to 6 days, up to 1 week, up to 2 weeks, up to 3 weeks, or more than 3 weeks following appearance of morphological features associated with aging. In some instances, the compositions described herein are administered immediately following appearance of morphological features associated with aging including up to 1 hour, up to 2 hours, up to 3 hours, up to 5 hours, up to 6 hours, up to 7 hours, up to 8 hours, up to 12 hours, up to 16 hours, up to 20 hours, or up to 24 hours following appearance of morphological features associated with aging. Sometimes the compositions described herein are administered singly, or over a time course, such as daily, multiple times weekly, weekly, biweekly, monthly or less frequently following appearance of morphological features associated with aging. In some instances, the compositions described herein are administered singly, or over a time course, such as daily, multiple times weekly, weekly, biweekly, monthly or more frequently following appearance of morphological features associated with aging. In some embodiments, the compositions are topical compositions. In some instances, the topical compositions are administered twice daily for at least or about 1 week, 2 weeks, 3 weeks, 1 month, 2 months, 3 months, 4 months, 5 months, 6 months, or more following appearance of morphological features associated with aging. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more following appearance of morphological features associated with aging.

Described herein are compositions and methods that can be administered to aging skin. In some instances, the aging skin is a region of the face, back of hands and nuchal region, forearm, dorsum of hand, presternal area, scalp and pretibial zones, calves, and combinations thereof.

A plurality of compositions may be used. For example, a first composition such as the composition of Table 1 or any component thereof or Table 2 or any component thereof is used in conjunction with a second composition such as the composition of Table 3 or any component thereof. In some instances, the first composition is administered before administration of the second composition. In some instances, the first composition is administered after administration of the second composition. In some instances, the first composition and the second composition are administered simultaneously. In some embodiments, a first composition is used for acute bruising or bleeding. In some embodiments, the first composition is used for acute episodic purpura. In some embodiments, the first composition is used for clearing the blood products, resolving bruising, decreasing inflammation, continuing the ECM recycling, or combinations thereof. In some embodiments, the first composition comprises tripeptide-1, tetrapeptide-2, hexapeptide-12, hexapeptide-11, and hexapeptide-38. In some embodiments, a second composition is used for maintaining and/or preventing bruising. In some embodiments, the second composition promotes elastin generation (e.g., elastin synthesis), promotes collagen synthesis, reduces skin flaccidity, reduces dermal disorganization, promotes LOXL synthesis, or combinations thereof. In some embodiments, the second composition comprises tripeptide-1, tetrapeptide-2, hexapeptide-12, and hexapeptide-11.

In some instances, the first composition is administered up to 1 day, up to 2 days, up to 3 days, up to 5 days, up to 6 days, up to 1 week, up to 2 weeks, up to 3 weeks, or more than 3 weeks prior to administration of the second composition. In some instances, the first composition is administered immediately prior to administration of the second composition including up to 1 hour, up to 2 hours, up to 3 hours, up to 5 hours, up to 6 hours, up to 7 hours, up to 8 hours, up to 12 hours, up to 16 hours, up to 20 hours, or up to 24 hours prior to the administration of the second composition. Sometimes the first composition is administered singly, or over a time course, such as daily, multiple times weekly, weekly, biweekly, monthly or less frequently prior to administration of the second composition.

In some instances, the first composition is administered up to 1 day, up to 2 days, up to 3 days, up to 5 days, up to 6 days, up to 1 week, up to 2 weeks, up to 3 weeks, or more than 3 weeks after the administration of the second composition. In some instances, the first composition is administered immediately after the administration of the second composition including up to 1 hour, up to 2 hours, up to 3 hours, up to 5 hours, up to 6 hours, up to 7 hours, up to 8 hours, up to 12 hours, up to 16 hours, up to 20 hours, or up to 24 hours after the administration of the second composition. Sometimes the first composition is administered singly, or over a time course, such as daily, multiple times weekly, weekly, biweekly, monthly or less frequently after the administration of the second composition.

### Stability Testing

Stability testing of the compositions can be conducted as follows.

High temperature testing is now commonly used as a predictor of long-term stability. High temperature testing can be conducted at 37° C. (98° F.) and 45° C. (113° F.). If a product is stored at 45° C. for three months (and exhibits acceptable stability) then it should be stable at room temperature for two years. A good control temperature is 4° C. (39° F.) where most products will exhibit excellent stability. Sometime, the product is also be subjected to −10° C. (14° F.) for three months.

In some instances, stability of the product is assessed by passing three cycles of temperature testing from −10° C. (14° F.) to 25° C. (77° F.). In such cases, the product is placed at −10° C. for 24 hours and then placed at room temperature (25° C.) for 24 hours. This completes one cycle. An even more rigorous test is a −10° C. to 45° C. five-cycle test. This puts emulsions under a tremendous stress.

The dispersed phase (of an oil-in-water emulsion) has a tendency to separate and rise to the top of the emulsion forming a layer of oil droplets. This phenomenon is called creaming. Creaming is one of the first signs of impending emulsion instability. A test method to predict creaming is centrifugation. Heat the emulsion to 50° C. (122° F.) and centrifuge it for thirty minutes at 3000 rpm. Then inspect the resultant product for signs of creaming.

Both formulas and packaging can be sensitive to the UV radiation. The product is placed in glass and the actual package in a light box that has a broad-spectrum output. Another glass jar completely covered with aluminum foil serves as a control. Discoloration of the product may be observed.

For all the above mentioned tests the color, odor/fragrance, viscosity, pH value, and, if available, particle size uniformity and/or particle agglomeration under the microscope can be ob served.

### Kits

Some embodiments of the methods and compositions provided herein include kits comprising peptides provided herein. In some embodiments, kits can be provided to an administering physician, other health care professional, a patient, or a caregiver. In some embodiments, a kit comprises a container which contains the peptide compositions in a suitable topical composition, and instructions for administering the peptide composition to a subject. The kit can optionally also contain one or more additional therapeutic or other agents. For example, a kit containing a peptide composition in topical form can be provided along with other skin care agents, such as, cleansers, occlusive moisturizers, penetrating moisturizers, sunscreens, sunblocks, and the like. The kit may contain the peptide composition in bulk form, or can contain separate doses of the peptide composition for serial or sequential administration. The kit can optionally contain one or more diagnostic tools, administration tools, and/or instructions for use. The kit can contain suitable delivery devices, such as, syringes, pump dispensers, single dose packets, and the like, along with instructions for administering the peptide compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

In some embodiments, the composition works with the skin's own natural regenerating process and assists in improving the skin's appearance, and skin tightness. The composition can increase natural levels of elastin in the skin, improves the quality of existing elastin, stimulates increase in collagen production, and exhibits high antioxidant activity to reduce inflammation, redness and irritation. The topical composition is suitable for all skin types and post-procedure skin. The topical compositions can be provided to the patient in bulk form, to permit a suitable amount of the peptides to be self-administered by the patient. For example, the patient can apply an amount of the composition sufficient to provide an even coating over the affected area or as otherwise instructed by the physician. In certain embodiments it can desirable to incorporate additional therapeutic or active agents into the topical composition. Alternatively, adjunct therapies or agents can be administered separately. For example, a cleanser, a sunblock, a sunscreen, a penetrating moisturizer, and/or an occlusive moisturizer can be provided for administration before or after the topical composition of the embodiments.

The kit may include a topical peptide composition, an occlusive moisturizer, a gentle cleanser, a penetrating moisturizer, and a broad spectrum SPF 30+ sunscreen.

The various examples of creams, ointments, lotions, solutions, gels, sprays and patches may incorporate the peptide compositions as described herein as the active ingredient, in combination with penetration enhancing agents and other active agents.

### Numbered Embodiments

Numbered embodiment 1 is a method for promoting restoration of aging skin comprising administering to a skin region of an individual with dermatoporosis a composition comprising a tripeptide-1, a hexapeptide-12, a hexapeptide-11, and a tetrapeptide-2. Numbered embodiment 2 is a method comprising numbered embodiment 1, wherein the tripeptide-1 is present at 1-10 ppm. Numbered embodiment 3 is a method comprising any one of numbered embodiments 1-2, wherein the tripeptide-1 is present at about 0.0001 wt. % to about 1 wt. %. Numbered embodiment 4 is a method comprising any one of numbered embodiments 1-3, wherein the tripeptide-1 is present at about 1 wt. % to about 6 wt. %. Numbered embodiment 5 is a method comprising any one of numbered embodiments 1-4 wherein the tripeptide-1 comprises palmitoyl tripeptide-1, myristoyl tripeptide-1, or a combination thereof. Numbered embodiment 6 is a method comprising any one of numbered embodiments 1-5 wherein, the hexapeptide-12 comprises palmitoyl hexapeptide-12, myristoyl hexapeptide-12, or a combination thereof. Numbered embodiment 7 is a method comprising any one of numbered embodiments 1-6 wherein, the hexapeptide-12 is present at 1-10 ppm. Numbered embodiment 8 is a method comprising any one of numbered embodiments 1-7 wherein, the hexapeptide-12 is present at about 0.0001 wt. % to about 1 wt. %. Numbered embodiment 9 is a method comprising any one of numbered embodiments 1-8 wherein, the hexapeptide-12 is present at about 0.25 wt. % to about 4 wt. %. Numbered embodiment 10 is a method comprising any one of numbered embodiments 1-9 wherein, the hexapeptide-11 is present at 50-150 ppm. Numbered embodiment 11 is a method comprising any one of numbered embodiments 1-10 wherein, the hexapeptide-11 is present at about 0.005 wt. % to about 0.02 wt. %. Numbered embodiment 12 is a method comprising any one of numbered embodiments 1-11 wherein, the composition further comprises hexapeptide-38. Numbered embodiment 13 is a method comprising any one of numbered embodiments 1-12 wherein, the hexapeptide-38 is acetyl hexapeptide-38. Numbered embodiment 14 is a method comprising any one of numbered embodiments 1-13 wherein, the hexapeptide-38 is present at about 0.0001 wt. % to about 1 wt. %. Numbered embodiment 15 is a method comprising any one of numbered embodiments 1-14 wherein, the method further comprises phosphatidylserine. Numbered embodiment 16 is a method comprising any one of numbered embodiments 1-15 wherein, the phosphatidylserine is present in a range of about 0.005 wt. % to about 0.1 wt. %. Numbered embodiment 17 is a method comprising any one of numbered embodiments 1-16 wherein, the phosphatidylserine is present at no more than 5.0 wt %. Numbered embodiment 18 is a method comprising any one of numbered embodiments 1-17 wherein, the composition further comprises hydroxymethoxyphenyl decanone. Numbered embodiment 19 is a method comprising any one of numbered embodiments 1-18 wherein, the composition further comprises dill extract. Numbered embodiment 20 is a method comprising any one of numbered embodiments 1-19 wherein, the composition further comprises palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica Montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or combinations thereof. Numbered embodiment 21 is a method comprising any one of numbered embodiments 1-20 wherein, the aging skin is more prone to bruising, is subject to photodamage, is exposed to solar radiation, is exposed to actinic damage, or combinations thereof. Numbered embodiment 22 is a method comprising any one of numbered embodiments 1-21 wherein, the composition is administered prior to appearance of a bruise, an aging spot, or a wrinkle. Numbered embodiment 23 is a method comprising any one of numbered embodiments 1-22 wherein, the composition is administered after appearance of a bruise, an aging spot, or a wrinkle. Numbered embodiment 24 is a method comprising any one of numbered embodiments 1-23 wherein, the composition is administered 1, 2, 3, 4, 5, 6, 7, or 8 times a day. Numbered embodiment 25 is a method comprising any one of numbered embodiments 1-24 wherein, the individual is a human. Numbered embodiment 26 is a method comprising any one of numbered embodiments 1-25 for promoting restoration of aging skin comprising administering to a skin region of an individual with dermatoporosis a first composition comprising a first tripeptide-1, a first hexapeptide-12, a first hexapeptide-11, and a first tetrapeptide-2, and a second composition comprising a second tripeptide-1, a second hexapeptide-12, a second hexapeptide-11, a second tetrapeptide-2, and a hexapeptide-38. Numbered embodiment 27 is a method comprising any one of numbered embodiments 1-26 wherein, the first tripeptide-1, the second tripeptide-1, or both is present at 1-10 ppm. Numbered embodiment 28 is a method comprising any one of numbered embodiments 1-27 wherein, the first tripeptide-1, the second tripeptide-1, or both is present at about 0.0001 wt. % to about 1 wt. %. Numbered embodiment 29 is a method comprising any one of numbered embodiments 1-28 wherein, the first tripeptide-1, the second tripeptide-1, or both is present at about 1 wt. % to about 6 wt. %. Numbered embodiment 30 is a method comprising any one of numbered embodiments 1-29 wherein, the first tripeptide-1, the second tripeptide-1, or both comprises palmitoyl tripeptide-1, myristoyl tripeptide-1, or a combination thereof. Numbered embodiment 31 is a method comprising any one of numbered embodiments 1-30 wherein, the first hexapeptide-12, the second hexapeptide-12, or both comprises palmitoyl hexapeptide-12, myristoyl hexapeptide-12, or a combination thereof. Numbered embodiment 32 is a method comprising any one of numbered embodiments 1-31 wherein, the first hexapeptide-12, the second hexapeptide-12, or both is present at 1-10 ppm. Numbered embodiment 33 is a method comprising any one of numbered embodiments 1-32 wherein, the first hexapeptide-12, the second hexapeptide-12, or both is present at about 0.0001 wt. % to about 1 wt. %. Numbered embodiment 34 is a method comprising any one of numbered embodiments 1-33 wherein, the first hexapeptide-12, the second hexapeptide-12, or both is present at about 0.25 wt. % to about 4 wt. %. Numbered embodiment 35 is a method comprising any one of numbered embodiments 1-34 wherein, the first hexapeptide-11, the second hexapeptide-11, or both is present at 50-150 ppm. Numbered embodiment 36 is a method comprising any one of numbered embodiments 1-35 wherein, the first hexapeptide-11, the second hexapeptide-11, or both is present at about 0.005 wt. % to about 0.02 wt. %. Numbered embodiment 37 is a method comprising any one of numbered embodiments 1-36 wherein, the hexapeptide-38 is present at about 0.0001 wt. % to about 1 wt. %. Numbered embodiment 38 is a method comprising any one of numbered embodiments 1-37 wherein, the first composition, the second composition, or both further comprises phosphatidylserine. Numbered embodiment 39 is a method comprising any one of numbered embodiments 1-38 wherein, the phosphatidylserine is present in a range of about 0.005 wt. % to about 0.1 wt. %. Numbered embodiment 40 is a method comprising any one of numbered embodiments 1-39 wherein, the phosphatidylserine is present at no more than 5.0 wt %. Numbered embodiment 41 is a method comprising any one of numbered embodiments 1-40 wherein, the first composition, the second composition, or both composition further comprises hydroxymethoxyphenyl decanone. Numbered embodiment 42 is a method comprising any one of numbered embodiments 1-41 wherein, the first composition further comprises dill extract. Numbered embodiment 43 is a method comprising any one of numbered embodiments 1-42 wherein, the first composition, the second composition, or both further comprises palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or combinations thereof. Numbered embodiment 44 is a method comprising any one of numbered embodiments 1-43 wherein, the aging skin is more prone to bruising, is subject to photodamage, is exposed to solar radiation, is exposed to actinic damage, or combinations thereof. Numbered embodiment 45 is a method comprising any one of numbered embodiments 1-44 wherein, the first composition, the second composition, or both is administered prior to appearance of a bruise, an aging spot, or a wrinkle. Numbered embodiment 46 is a method comprising any one of numbered embodiments 1-45 wherein, the first composition, the second composition, or both is administered after appearance of a bruise, an aging spot, or a wrinkle. Numbered embodiment 47 is a method comprising any one of numbered embodiments 1-46 wherein, the first composition is administered prior to appearance of a bruise, an aging spot, or a wrinkle and the second composition is administered after appearance of the bruise, the aging spot, or the wrinkle. Numbered embodiment 48 is a method comprising any one of numbered embodiments 1-47 wherein, the first composition, the second composition, or both is administered 1, 2, 3, 4, 5, 6, 7, or 8 times a day. Numbered embodiment 49 is a method comprising any one of numbered embodiments 1-48 wherein, the individual is a human.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1

### Exemplary Compositions

Exemplary compositions are seen in Tables 1-3.

TABLE 1

| Ingredient | % by wt. |
|---|---|
| Butylene Glycol, Aqua, Acetyl Hexapeptide-38 | 0.05-1.25 |
| Xylitylglucoside, Anhydroxylitol, Xylitol | 0.2-5 |
| Water, Butylene Glycol, Arnica Montana Flower Extract | 0.1-2.5 |
| Glycerin, Palmitoyl Tripeptide-1 | 0.5-15 |
| Glycerin, Palmitoyl Hexapeptide-12 | 0.5-15 |
| Hexapeptide-11 | 0.001-0.025 |
| Sodium Hyaluronate Crosspolymer | 0.1-2.5 |
| Squalane, Dunaliella Salina Extract | 0.1-2.5 |
| Ledum Palustre (Labrador Tea) Extract, Radish Root | 0.1-2.5 |
| Ferment Filtrate | |
| Lactoferrin | 0.01-0.25 |
| Phosphatidylserine, Phospholipids, Tocopherol, Ascorbyl | 0.02-0.5 |
| Palmitate | |
| Aqua, Butylene Glycol, Peucedanum Graveolens (Dill) | 0.1-2.5 |
| Extract, Xanthan Gum | |
| Water, Tremella Fuciformis Sporocarp (Silver Ear | 0.1-2.5 |
| Mushroom) Extract, Betaine, Glycerin | |
| Propanediol, Lecithin | 0.4-10 |
| Caprylic/Capric Triglyceride, Hydroxymethoxyphenyl | 0.1-2.5 |
| Decanone | |
| Water, Acetyl Tetrapeptide-2, Caprylyl Glycol | 0.2-5 |
| Caprylic/Capric Triglyceride | 0.4-10 |
| Caprylyl Methicone | 0.1-2.5 |
| Water/Aqua/Eau | 40-90 |
| Phenoxyethanol, Ethylhexylglycerin | 0.17-4.25 |
| Polyacrylate-13, Polyisobutene, Polysorbate 20 | 0.5-12.5 |
| Caprylyl Glycol, Caprylhydroxamic Acid, Glycerin | 0.1-2.5 |
| Disodium EDTA | 0.02-0.5 |
| Propanediol | 0.04-1 |

TABLE 2

| Ingredient | % by wt. |
|---|---|
| Water/Aqua/Eau, | 50-95 |
| Glycerin | 0.5-9 |
| Caprylic/Capric Triglyceride | 1-9 |
| Propanediol | 0.01-5 |
| Polyacrylate-13 | 0.5-6 |
| Lactoferrin | 0.01-1 |
| Phosphatidylserine | 0.01-1 |
| Ledum Palustre (Labrador Tea) Extract | 0.1-2.5 |
| Arnica Montana Flower Extract | 0.0001-1 |
| Palmitoyl Hexapeptide-12 | 0.0001-1 |
| Palmitoyl Tripeptide-1 | 0.0001-1 |
| Hexapeptide-11 | 0.00500 |
| Acetyl Hexapeptide-38 | 0.0001-1 |
| Acetyl Tetrapeptide-2 | 0.0001-1 |
| Sodium Hyaluronate Crosspolymer | 0.0001-2.5 |
| Tremella Fuciformis Sporocarp (Silver Ear | 0.001-2.5 |
| Mushroom) Extract | |
| Peucedanum Graveolens (Dill) Extract | 0.01-2.5 |
| Hydroxymethoxyphenyl Decanone | 0.001-0.1 |
| Dunaliella Salina Extract | 0.001-0.5 |
| Betaine | 0.01-0.5 |
| Phospholipids | 0.01-1 |
| Xylitylglucoside | 0.1-2 |
| Squalane | 0.1-0.8 |
| Caprylyl Glycol | 0.1-0.5 |
| Anhydroxylitol | 0.1-1.5 |
| Polysorbate 20 | 0.01-0.5 |
| Xylitol | 0.1-0.5 |
| Butylene Glycol | 0.1-2 |
| Sorbitan Isostearate | 0.1-1 |
| Ethylhexylglycerin | 0.01-1 |
| Caprylhydroxamic Acid | 0.05-0.5 |
| Ascorbyl Palmitate | 0.001-0.1 |
| Xanthan Gum | 0.01-0.8 |
| Pentylene Glycol | 0.01-0.8 |
| Glucose | 0.01-0.8 |
| Helianthus Annuus (Sunflower) Seed Oil | 0.001-0.5 |
| Tocopherol | 0.001-0.8 |
| Leuconostoc/Radish Root Ferment Filtrate | 0.01-0.8 |
| Potassium Sorbate | 0.001-0.5 |
| Caprylyl Methicone | 0.1-0.8 |
| Polyisobutene | 0.1-.8 |
| Lecithin | 0.1-1 |
| Disodium EDTA | 0.1-1 |
| Phenoxyethanol | 0.1-2 |

TABLE 3

| Ingredient | % by wt. |
|---|---|
| Hydrogenated Lecithin, | 1-6% |
| C12-16 Alcohols, Palmitic Acid | |
| Avocado extract, Shea | 0.25-2% |
| butter, Bentonite | |
| Acetyl Tetrapeptide-2 | 1-4% |
| Phytoene/Phytofluene | 0.2-1% |
| Hydroxymethoxyphenyl | 0.5-2% |
| Decanone- | |
| TriHex -Palmitoyl | 1-6% |
| Tripeptide-1 | |
| Palmitoyl Hexapeptide -12 | 0.25-4% |
| Polyholosides from flax seeds | 2.5-10% |
| Plantago lanceolata also | 1-4% |
| called “Plantain” | |
| Dill extract | 0.25-4% |
| Phosphatidylserine | 0.025-0.1% |
| Oleuropein | 0.01-0.05% |
| Hexapeptide-11 | 0.005-0.02% |
| Hydrolyzed Candida | 1-6% |
| Saitoana Extract | |
| Centella Asiatica | 0.25-4% |
| Propanediol, Lecithin | 0.25-2% |
| Euglena Gracilis Extract, | 0.05-1% |
| Aqua, Caffeine, Glaucium | |
| Flavum Leaf Extract | |

### Example 2

### In Vivo Testing of Bruising Resolution

A randomized, double-blind study was undertaken to assess the efficacy and safety of a topical product formulated to increase the elimination of blood products that manifest as a bruise. This study investigated the ability of the topical product comprising a formula as described in Table 2 to improve the appearance of a bruise compared to a bland moisturizer.

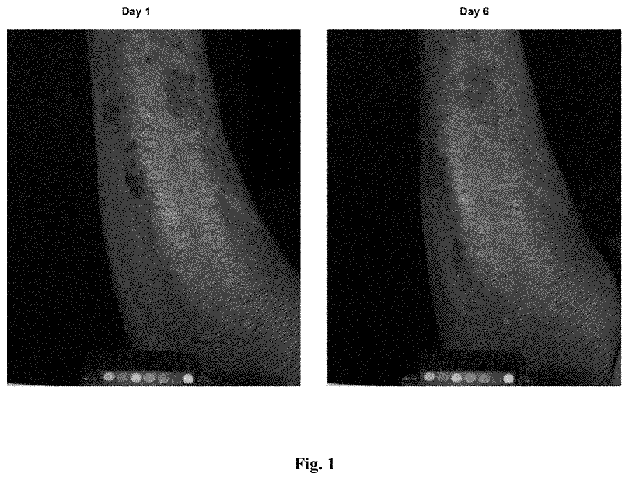

A formula as described in Table 2 was applied. After day 6, application of the topical product reduced senile purpura as seen in FIG. 1.

The data demonstrate improvements in bruising resolution using the topical product.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for promoting restoration of aging skin comprising administering to a skin region of an individual with dermatoporosis a first composition comprising:
a first tripeptide-1;
a first hexapeptide-12;
a first hexapeptide-11; and
a first tetrapeptide-2.

2. The method of claim 1, wherein the first tripeptide-1 is present at:
about 0.0001 wt. % to about 1 wt. %; or
1-10 ppm.

3. The method of claim 1, wherein the first hexapeptide-12 is present at:
about 0.0001 wt. % to about 1 wt. %; or
about 1-10 ppm.

4. The method of claim 1, wherein the first hexapeptide-11 is present at:
about 0.005 wt. % to about 0.02 wt. %; or
about 50-150 ppm.

5. The method of claim 1, wherein the first tetrapeptide-2 is present at about 0.25 wt. % to about 10 wt. %.

6. The method of claim 1, wherein the first composition further comprises a hexapeptide-38, wherein the hexapeptide-38 is present in the first composition at about 0.0001 wt. % to about 1 wt. %.

7. The method of claim 1, wherein the first composition comprises phosphatidylserine, wherein the phosphatidylserine is present in the first composition at no more than 5.0 wt %.

8. The method of claim 1, wherein the first composition further comprises hydroxymethoxyphenyl decanone, dill extract, palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or a combination thereof.

9. The method of claim 1, wherein the aging skin is prone to bruising, is subject to photodamage, is exposed to solar radiation, is exposed to actinic damage, or combinations thereof.

10. The method of claim 1, further comprising administering to the skin region of the individual with dermatoporosis a second composition comprising a second tripeptide-1, a second hexapeptide-12, a second hexapeptide-11, a second tetrapeptide-2, and a hexapeptide-38.

11. The method of claim 10, wherein the second tripeptide-1 is present at:
about 0.0001 wt. % to about 1 wt. %; or
1-10 ppm.

12. The method of claim 10, wherein the second hexapeptide-12 is present at:
about 0.0001 wt. % to about 1 wt. %; or
about 1-10 ppm.

13. The method of claim 10, wherein the second hexapeptide-11 is present at:
about 0.005 wt. % to about 0.02 wt. %; or
about 50-150 ppm.

14. The method of claim 10, wherein the second tetrapeptide-2 is present at about 0.25 wt. % to about 10 wt. %.

15. The method of claim 10, wherein the hexapeptide-38 is present in the second composition at about 0.0001 wt. % to about 1 wt. %.

16. The method of claim 10, wherein the second composition comprises phosphatidylserine, wherein the phosphatidylserine is present in the second composition at no more than 5.0 wt %.

17. The method of claim 10, wherein the second composition further comprises hydroxymethoxyphenyl decanone, dill extract, palustre extract, Tremella fuciformis extract, butylene glycol, glycerin, squalane, Dunaliella salina extract, phospholipids, tocopherol, ascorbyl palmitate, xanthan gum, betaine, propanediol, lecithin, caprylic/capric triglyceride, caprylyl glycol, caprylyl methicone, phenoxyethanol, ethylhexylglycerin, polyacrylate-13, polyisobutene, polysorbate 20, caprylhydroxamic acid, disodium EDTA, Arnica montana extract, sorbitan isostearate, pentylene glycol, glucose, sunflower seed oil, radish root ferment filtrate, potassium sorbate, sodium hyaluronate crosspolymer, xylitylglucoside, anhydroxylitol, xylitol, or a combination thereof.

18. The method of claim 10, wherein the first composition is administered prior to appearance of a bruise, an aging spot, or a wrinkle.

19. The method of claim 10, wherein the second composition is administered after appearance of a bruise, an aging spot, or a wrinkle.

20. The method of claim 10, wherein the first composition is administered prior to appearance of a bruise, an aging spot, or a wrinkle, and the second composition is administered after appearance of the bruise, the aging spot, or the wrinkle.
